(19) United States
(12) Patent Application Publication
(10) Pub. No.: US 2009/0209460 A1
(43) Pub. Date: Aug. 20, 2009

(54) COMPOSITIONS AND METHODS FOR TREATING OBESITY AND RELATED METABOLIC DISORDERS

(75) Inventors: Andrew A Young, Rancho Santa Fe, CA (US); Sarah L. McQuaid, San Diego, CA (US); Richard A. Pittner, San Diego, CA (US); Ved Srivastava, San Diego, CA (US)

Correspondence Address: Intellectual Property Department;Amylin Pharmaceuticals, Inc., 9360 Towne Centre Drive, San Diego, CA, 92121, US

(73) Assignee: Amylin Pharmaceuticals, Inc., San Diego (CA)

(21) Appl. No.: 12/097,737

(22) PCT Filed: Dec. 15, 2006
(86) PCT No.: PCT/US2006/047953

§ 371 Date: Oct. 14, 2008

Related U.S. Application Data
(60) Provisional application No. 60/751,412, filed on Dec. 16, 2005.

(51) Int. Cl. A61K 38/16; C07K 7/08; A61K 38/10; C07K 14/00; A61P 3/10

(52) U.S. Cl. 514/12; 530/326; 514/14; 530/324

(57) ABSTRACT

The present invention relates to the use of neuromedins in methods to treat and prevent conditions such as obesity and other food-related disorders. In addition, novel peptides, FNX Peptides, are provided, which find use in treating these disorders.

### CROSS-REFERENCE APPLICATIONS

This is a national stage filing of PCT/US2006/047953 which claims priority to U.S. Ser. No. 60/751,412, filed Dec. 16, 2005, the entirety of which is hereby incorporated by reference. Also incorporated by reference in their entirety for NMU and FNX compounds are commonly-owned WO2005/077072, PCT/US2006/031724 filed Aug. 11, 2006, WO2006/86769 and U.S. Ser. No. 11/206,903, filed Aug. 17, 2005. An electronic copy of the Sequence Listing in computer readable form is hereby incorporated by reference.

### FIELD

The present application is directed to the use of neuromedin compounds for treating or preventing conditions such as obesity and related metabolic disorders and conditions. More specifically, the condition or disorder can be one in which the reduction of food or caloric intake is of value, e.g. being undesirably overweight, eating disorders, metabolic syndrome. The present application is further directed to novel neuromedin compounds for treating or preventing conditions such as obesity and related metabolic disorders.

### BACKGROUND

Obesity is a condition that affects millions of Americans. Recent statistics by the Center for Disease Control (“CDC”) estimate that approximately 65% of all Americans are overweight or obese and it is generally believed that these numbers are increasing. Being obese or overweight may substantially increase the risk of morbidity from numerous other conditions. Higher body weights are also associated with increases in all-cause mortality. Furthermore, being obese or overweight may cause a person to have negative self-image.

In humans, patients who are overweight or obese are considered those with a Body Mass Index (BMI) of equal or greater than 25. BMI, a common measure expressing the relationship (or ratio) of weight-to-height, is calculated by dividing a person's body weight in kilograms by the square of the person's height in meters (i.e., wt/(ht)2). Individuals with a BMI of 25 to 29.9 are considered overweight, while individuals with a BMI of 30 or more are considered obese.

According to the NIH Clinical Guidelines on the Identification, Evaluation, and Treatment of Overweight and Obesity in Adults, all adults (aged 18 years or older) who have a BMI of 25 or more are considered at risk for premature death and disability as a consequence of being overweight or obese. These health risks further increase as the severity of an individual's obesity increases.

Hypernutrition is also the result of, and the psychological cause of, many eating disorders, such as diet-induced obesity. Reducing food intake would be beneficial in the treatment of such disorders. At least three distinctive eating patterns have been reported, night-eating syndrome (characterized by morning anorexia, evening hyperphagia, and insomnia), eating binge (in which large amounts of food are consumed in an orgiastic manner at irregular intervals) and eating-without-satiation (which has been observed in persons suffering from damage to the central nervous system).

Metabolic Syndrome has as one underlying factor obesity, particularly abdominal obesity (which presents clinically as increased waist circumference), that can arise from or worsen by overeating. The “obesity epidemic” has been cited as mainly responsible for the rising prevalence of metabolic syndrome.

For these reasons, there is an enormous interest in treating obesity and related metabolic disorders. Existing therapies include standard diets and exercise, very low calorie diets, behavioral therapy, pharmacotherapy involving appetite suppressants, thermogenic drugs, food absorption inhibitors, mechanical devices such as jaw wiring, waist cords and balloons, and surgery, such as gastric bypass. Jung and Chong, Clinical Endocrinology, 35:11-20 (1991); Bray, Am. J. Clin. Nutr., 55:538 S-544S (1992). However, additional methods for reducing weight or treating obesity are still needed.

### SUMMARY

Provided are methods and compositions useful in the control, treatment and prevention of obesity and eating disorders and related conditions and disorders. In one embodiment is a method of decreasing food intake or body weight of a subject that comprises administering to the subject an effective amount of an NMX Peptide, FNX Peptide or NMX Receptor agonist. In certain embodiments are provided methods of treating or preventing a condition or disease that can be alleviated by reducing caloric or nutrient intake or availability in a subject. Such conditions and diseases include but are not limited to obesity, metabolic syndrome and obesity-related diabetes mellitus. In other embodiments are provided methods for the control, prevention or treatment of conditions or disorders associated with eating, such as binge eating, food cravings, and stress-induced or -associated food disorders, as by controlling food intake for example. In one embodiment the NMX Peptide, FNX Peptide or NMX Receptor agonist is administered systemically, and in another the compounds are administered, e.g. locally, to provide delivery to the gut, which as described herein may provide a distension signal inducing satiety.

In one embodiment, the NMX Peptide, FNX Peptide or NMX Receptor agonist is co-administered with at least one other obesity-reducing compound. Such a drug can mediate decreased food intake or reduce body weight or induce satiety, by any of a number of means, including, but not limited to, suppressing hunger, controlling appetite, increasing metabolism, etc. The at least one other drug may cause weight loss. The at least one other drug can be administered as a bolus dose or as a continuous dose.

In a further embodiment a method of reducing caloric intake in a subject is provided, wherein the method comprises administering an effective amount of NMX Peptide, FNX Peptide or NMX Receptor agonist to said subject as a replacement for a meal or snack.

In yet a further embodiment a method of reducing caloric intake by reducing the size of a meal is provided, wherein the method comprises administering an effective amount of NMX Peptide, FNX Peptide or NMX Receptor agonist to the subject.

In yet a further embodiment a method of controlling food intake is provided, wherein the method comprises administering an effective amount of NMX Peptide, FNX Peptide or NMX Receptor agonist to said subject.

In yet another embodiment a method for ensuring or assisting in compliance with a reduced calorie or restrictive diet or diet plan is provided, wherein the method comprises administering an effective amount of NMX Peptide, FNX Peptide or NMX Receptor agonist to said subject.

In yet a further embodiment a method of maintaining weight loss or maintaining the weight lost is provided, wherein the method comprises administering an effective amount of NMX Peptide, FNX Peptide or NMX Receptor agonist to said subject.

Also provided is a method of controlling caloric intake in a subject, wherein the method comprises administering to the subject an effective amount of NMX Peptide, FNX Peptide or NMX Receptor agonist at particular times of the day when the subject is more likely to overeat or eat palatable, sweet or savory foods.

In one embodiment is provided a composition comprising an NMX Peptide, FNX Peptide or NMX Receptor agonist, optionally with the at least one other anti-obesity drug, and a pharmaceutically acceptable carrier. The composition can be contained in a kit comprising one dosage form of an NMX Peptide, FNX Peptide or NMX Receptor agonist, optionally with a second dosage form comprising the at least one other drug.

In further embodiments, any of the methods disclosed herein result in the subject's body weight being reduced by at least 1% to at least 50%. In additional embodiments, any of the methods disclosed herein result in the subject's body weight being reduced by at least about 5 pounds or 2 kg, to at least about 200 pounds or 100 kg. In still further embodiments, practice of any of the methods disclosed herein results in weight reduction, wherein less than about 40% to less than about 1%, or 0% of the weight loss is due to loss of mean body mass.

In additional embodiments the subject has a body mass index (BMI) of greater than or equal to about 25, while in other embodiments the subject has a BMI of greater than or equal to about 30. In other embodiments the subject suffers from diabetes, insulin resistance or impaired glucose tolerance, while in other embodiments the subject does not suffer from diabetes, insulin resistance, metabolic syndrome, or impaired glucose tolerance.

Also provided are novel FNX Peptides. In one embodiment novel FNX Peptides comprise an amino acid sequence of formula (I): F1-P, where F1-P is a novel non-naturally occurring combination of an F1 and P segments, where P is an octapeptide as described herein capable of providing, when attached to the F1 portion and systemically delivered, suppression of food intake, reduction of body weight, and/or induction of a satiety signal, and wherein F1 is a des-octapeptide portion of an FN38 or analog or derivative or chimera thereof, as described herein, which enhances or enables P activity. Additional octapeptides and F1 portions are disclosed herein, as well as methods to make and identify additional FNX Peptides. Excluded from F1-P are the FN38/36 compounds, GenBank Accession Number AJ510133 (human), CAD52851 (rat), CAD52850 (frog) and chicken FN38, however their respective F and P segments can be used to create the novel FNX Peptides described herein.

In another embodiment novel FNX Peptides comprise an amino acid sequence of formula (II): F2-P, where P is an octapeptide as described herein capable of providing, when attached to the F2 portion and systemically delivered, suppression of food intake, reduction of body weight, and/or induction of a satiety signal, and wherein F2 is a des-octapeptide portion of an FN38 and SN23 chimera or analog or derivative thereof, as described herein, which enhances or enables P activity. An exemplary effective hybrid is FN38(1-15)-SN23 (FLFHYSKTQKLGKSNSDEEVQVPGGVISNGYFLFRPRN-NH2; SEQ ID NO. 2), which is a hybrid of tree frog SN-23 NMU (SDEEVQVPGGVISNGYFLFRPRN-NH2; SEQ ID NO. 3) and human FN38 (FLFHYSKTQKLGKSNVVEELQSPFASQSRGYFLFRPRN-NH2 (SEQ ID NO. 4), additionally in its amide form (the “-NH2” indicating a C-terminal amide). In this embodiment the “P” octapeptide is YFLFRPRN (SEQ ID NO. 5) and the “F” portion is FLFHYSKTQKLGKSNSDEEVQVPGGVISNG (SEQ ID NO. 6). Additional octapeptides and F2 portions are disclosed herein, as well as methods to make and identify additional FNX Peptides

### BRIEF DESCRIPTION OF THE DRAWINGS

FIGS. 1A, 1B, and 1C show a comparison of in vivo potencies of rat NMU, FN-38 and SN-23 on suppression of food intake, at 30 minutes.

FIGS. 2A, 2B and 2C show a dose response of FN-38 on food intake measured at 30, 60 and 120 minutes.

FIG. 3 shows a comparison of anorectic effects of rat NMU-23, U-8 (porcine) and U-9 (rat).

FIGS. 4A, 4B and 4C depict the effect of FN38 to inhibit food intake in over-night fasted rats when given intraperitoneally.

FIG. 5 depicts the reduction of cumulative weight gain by peripheral, long term administration of FN38 amide and rat NMU-23 amide in rats with diet induced obesity (rat DIO).

FIGS. 6A and B depict the reduction of cumulative weight gain by peripherally administered FN38 analogs in mice with diet induced obesity (mouse DIO).

FIGS. 7A and 7B depict the reduction of cumulative weight gain by peripherally administered FN38 analogs in mice with diet induced obesity (mouse DIO).

### DETAILED DESCRIPTION

The applicant has discovered that certain NMX Peptides, FNX Peptides and NMX Receptor agonists, in contrast to literature reports, are very active in reducing food intake, caloric intake and body weight when systemically delivered, and that such NMX Peptides, FNX Peptides and NMX Receptor agonists are active systemically in contrast to the shorter neuromedins U8 or U9. Based upon the pharmacological activities described herein, polypeptides comprising FNX Peptides can be useful to treat conditions or disorders which can be alleviated by inhibiting or reducing food, caloric or nutrient intake or availability or by reducing or suppressing appetite. This includes any condition or disorder in a subject that is either caused by, complicated by, or aggravated by a relatively high food, caloric or nutrient intake or availability, or that can be alleviated by reducing food, caloric or nutrient intake or availability. Such conditions or disorders include, but are not limited to, hypernutrition, obesity, obesity-related diabetes mellitus, including type 2 diabetes associated with obesity, eating disorders (e.g. binge eating, bulimia nervosa, food cravings, stress-induced eating disorder), and insulin-resistance syndromes (e.g. metabolic syndrome X) associated with obesity. Accordingly provided are methods and compositions useful in the control, treatment and prevention of obesity and eating disorders and related conditions and disorders.

Applicants were the first to identify that FN38 and neuromedin U and S peptides can be used in the regulation of food intake when systemically administered in a mammal. Upon systemic administration of FN38 and NMU to mice, there was a suppression of food intake and reduction in body weight. Previous reports have indicated only that central administration (ICV) of NMU reduced food intake in rats, reported as an action of NMU on cerebrally located NMU receptors. In addition there have been no reports identifying an NMU as a circulating ligand. Nor is there any report suggesting activity for FN38 variants, i.e. GenBank Accession Number AJ510133. At this time, a physiologic role of NMU has not been reported.

A 327 bp partial mRNA for human neuromedin U (NMU gene), 38C isoform, was deposited as GenBank Accession Number AJ510133, reportedly as an alternative splice variant of human neuromedin U gene reportedly directly submitted on Oct. 2, 2002. From this mRNA's open reading frame, a 109 amino acid protein was proposed and deposited in Genbank as CAD52852, reportedly on Oct. 2, 2002. The deposit noted a mature peptide at positions 65 to 102, which yields sequence: FLFHYSKTQKLGKSNVVEEFQSPFASQSRGYFLFRPRNGRRSAGF (SEQ ID NO.: 1).

An alignment (using NCBI BLASTP 2.2.12 with default parameters) with applicant's novel FN38 peptide yields 97% identify (37/38) with the difference being the L20F substitution.

Also reported by the same authors were a 36 amino acid rat variant CAD52851 that is 76% identical to FN-38 and a green tree frog variant CAD52850 that is 60% identical. FN38 has the sequence: FLFHYSKTQKLGKSNVVEELQSPFASQSRGYFLFRPRN-NH2 (SEQ ID NO.: 4). CAD52851 rat has the sequence FLFHYSKTQKLGNSNVV-EYQGPVAP-SGGFFLFRPRN-NH2 (SEQ ID NO.: 7). A reported chicken variant has the sequence FLFHYSKTHDSGNSDVREDLQGTGGIQSRGYFFFRPRN-NH2 (SEQ ID NO.: 8). And the CAD52850 (tree frog) sequence is FLFHYSKSHDSGNSDITEEVQVPGGVISNGYFLFRPRN-NH2 (SEQ ID NO.: 9).

The role of the gut in regulating food intake is thought to involve two types of signals: the degree of distension of the gut and the activation of chemoreceptors in the gastric or intestinal wall. The gut is the largest endocrine organ in the body and after a meal a large number of gastrointestinal hormones are released. Some examples are gastrin, somatostatin, cholecystokinin, gastric inhibitory polypeptide and neurotensin. While not to be bound by theory, applicant believes that the NMX Peptides, FNX Peptides and NMX Receptor agonists can, in one embodiment, provide or mimic a signal indicating distension of the stomach, which leads to an increased satiety effect. Accordingly, in one embodiment NMX Peptides, FNX Peptides and NMX Receptor agonists provide a circulating distension signal, which has not been previously identified, and find use in increasing or inducing satiety or reducing food intake and caloric consumption. Artificial distension signals have been reported as effective aids to weight reduction. For example, in morbidly obese subjects with a mean excess weight of 51.3 kg, an implantable vagal stimulator has been associated with 23.8±5.0% weight loss over 10 months (Favretti et al. 2004). This degree of weight loss (12.2 kg) exceeds that usually attainable with current pharmacotherapy, but is less than the 70% loss of excess weight after bariatric surgery. Accordingly, NMX Peptides, FNX Peptides and NMX Receptor agonists acting as circulating distension signals represent a represent a novel mode of anti-obesity therapy, and particularly when combined with other therapeutics such as gut-peptide mimetics to synergize or enhance their artificial nutrient signals. NMX Peptides, FNX Peptides and NMX Receptor (NMU1R and NMU2R) agonists, thus provide beneficial regulation of obesity, food intake disorders and related disorders and conditions as discussed herein, and in further embodiments, they do so when systemically administered. In a further embodiment, the FN38 compounds find use when co-administered with drugs that affect nutrient signals, such as GLP-1, GIP, leptin and amylin and their mimetics.

The NMX Peptides, FNX Peptides and NMX Receptor agonists find use in inducing or enhancing satiety. The NMX Peptides, FNX Peptides and NMX Receptor agonists find further use to control body weight, and/or to control calorie intake and/or aid in adherence to a dietary plan, such as one intended to reduce or control or maintain body weight. For example, a subject following that plan may be better able to reduce, control or maintain their body weight. The term “dietary plan” as used herein includes those for controlling body weight and those followed for medical reasons.

Accordingly, selectively modulating NMU receptors systemically or by localized delivery to the gut can provide an approach to treatment of human obesity and other eating disorders. Identification of weight-regulating therapeutics (agonists) that modulate an NMU receptor (e.g., NMUR1, NMUR2) can lead to new drugs for the treatment of obesity and other weight disorders. Such agonists would be useful in the treatment, control or prevention of obesity (by reducing appetite, increasing satiety, reducing fat intake and/or reducing carbohydrate craving) and other disorders affected by the intake of food.

Further Uses of NMX Peptides, FNX Peptides or NMX Receptor Agonists. In one embodiment is a method of decreasing food intake or body weight of a subject that comprises administering to the subject an effective amount of an NMX Peptide, FNX Peptide or NMX Receptor agonist. In certain embodiments are provided methods of treating or preventing a condition or disease that can be alleviated by reducing caloric or nutrient intake or availability in a subject. Such conditions and diseases include but are not limited to obesity, metabolic syndrome and obesity-related diabetes mellitus. In other embodiments are provided methods for the control, prevention or treatment of conditions or disorders associated with eating, such as binge eating, food cravings, and stress-induced or -associated food disorders, as for example, by controlling food intake. In one embodiment the NMX Peptide, FNX Peptide or NMX Receptor agonist is administered systemically, and in another the compounds are administered, e.g. locally, to provide delivery to the gut, which as described herein may provide a distension signal inducing satiety. Accordingly exemplary modes of delivery include peripheral injection, infusion, absorption (e.g., mucosal, transmucosal, transdermal), oral, suppository, and inhalation, as well as gene therapy approaches using nucleic acids that encode the amino acid sequences described herein, and additionally, those that provide systemic delivery or a delivery targeted to the gut.

In a further embodiment a method of reducing caloric intake in a subject is provided, wherein the method comprises administering an effective amount of NMX Peptide, FNX Peptide or NMX Receptor agonist to said subject as a replacement for a meal or snack.

In yet a further embodiment a method of reducing caloric intake by reducing the size of a meal is provided, wherein the method comprises administering an effective amount of NMX Peptide, FNX Peptide or NMX Receptor agonist to the subject.

In yet a further embodiment a method of controlling food intake is provided, wherein the method comprises administering an effective amount of NMX Peptide, FNX Peptide or NMX Receptor agonist to said subject.

In yet another embodiment a method for ensuring or assisting in compliance with a reduced calorie or restrictive diet or diet plan is provided, wherein the method comprises administering an effective amount of NMX Peptide, FNX Peptide or NMX Receptor agonist to said subject.

In yet a further embodiment a method of maintaining weight loss or maintaining the weight lost is provided, wherein the method comprises administering an effective amount of NMX Peptide, FNX Peptide or NMX Receptor agonist to said subject.

In yet further embodiments are provided methods for controlling or modifying eating disorders are provided, wherein the methods comprise administering to a subject in need thereof an NMX Peptide, FNX Peptide or NMX Receptor agonist in an amount effect to control, curb or modify an eating disorder by the subject. The eating disorder includes hypernutrition, night-eating syndrome, binge eating and eating-without-satiation. By reducing or inhibiting food, caloric or nutrient intake or availability or by suppressing or reducing appetite, the methods provided herein provide therapeutic and/or desirous effects on eating disorders for the subject.

Also provided is a method of controlling caloric intake in a subject, wherein the method comprises administering to the subject an effective amount of NMX Peptide, FNX Peptide or NMX Receptor agonist at particular times of the day when the subject is more likely to overeat or eat palatable, sweet or savory foods.

In further embodiments, any of the methods disclosed herein result in the subject's body weight being reduced by at least 1% to at least 50%. In additional embodiments, any of the methods disclosed herein result in the subject's body weight being reduced by at least about 5 pounds or 2 kg, to at least about 200 pounds or 100 kg. In still further embodiments, practice of any of the methods disclosed herein results in weight reduction, wherein less than about 40% to less than about 1%, or 0% of the weight loss is due to loss of mean body mass.

In additional embodiments the subject has a body mass index (BMI) of greater than or equal to about 25, while in other embodiments the subject has a BMI of greater than or equal to about 30. In other embodiments the subject suffers from diabetes, insulin resistance or impaired glucose tolerance, while in other embodiments the subject does not suffer from diabetes, insulin resistance or impaired glucose tolerance.

In one embodiment is provided a composition comprising an NMX Peptide, FNX Peptide or NMX Receptor agonist, optionally with at least one other anti-obesity drug, and a pharmaceutically acceptable carrier. The composition can be contained in a kit comprising one or more dosage forms of an NMX Peptide, FNX Peptide or NMX Receptor agonist, optionally with a one or more dosage forms comprising the at least one other drug. A dosage form may be individual or multi-dose.

Given the biological activity and/or receptor binding activity described herein, the present invention provides NMX Peptides, FNX Peptides or NMX Receptor Agonists compositions for use in a medicament for treating a disease or disorder in a subject in need thereof. The present invention also provides methods for use of NMX Peptides, FNX Peptides or NMX Receptor Agonists compositions in treating a disease or disorder in a subject, such as those disclosed herein.

As used herein, and as well-understood in the art, “treatment” is an approach for obtaining beneficial or desired results, including clinical results. “Treating” a disease, disorder, or condition means that the extent and/or undesirable clinical manifestations of a condition, disorder, or a disease state are lessened and/or time course of the progression is slowed or lengthened, or prevented, as compared to not treating the disorder. For example, in treating obesity, a decrease in body weight, e.g., a 5% decrease in body weight, is an example of a desirable treatment result. For purposes of this invention, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Further, treating does not necessarily occur by administration of one dose, but often occurs upon administration of a series of doses. Thus, a therapeutically effective amount or an amount sufficient to treat a disease, disorder, or condition may be administered in one or more administrations.

In one embodiment, as used herein in the context of weight reduction, a “subject in need thereof” is a subject who is overweight or obese or afflicted with a condition or disorder described herein, or otherwise seeks to control caloric intake. In one embodiment, the subject is an obese or overweight subject. In exemplary embodiments, an “overweight subject” refers to a subject with a body mass index (BMI) greater than 25, or a BMI between 25 and 30. It should be recognized, however, that meaning of overweight is not limited to individuals with a BMI of greater than 25, but refers to any subject where weight loss is desirable for medical or cosmetic reasons. While “obesity” is generally defined as a body mass index over 30, for purposes of this disclosure, any subject, who needs or wishes to reduce body weight is included in the scope of “obese.” In one embodiment, subjects who are insulin resistant, glucose intolerant, or have any form of diabetes mellitus (e.g., type 1, 2 or gestational diabetes) can benefit from this method. In another embodiment, a subject in need thereof is obese. In still another embodiment, the subject has diabetes mellitus. A subject having diabetes mellitus may have type 1 or type 2 diabetes. It should be noted, however, that the method described herein may be applied to subjects who do not have and/or have not been diagnosed with impaired glucose tolerance, metabolic syndrome, insulin resistance or diabetes mellitus.

As such, in one aspect, the present invention provides NMX Peptides, FNX Peptides or NMX Receptor Agonists compositions and methods of using them to reduce weight in a subject; treat diabetes, including type 2 or non-insulin dependent diabetes, type 1 diabetes; and to treat eating disorders, insulin-resistance syndrome, and/or metabolic syndrome.

In one embodiment, methods for reducing body weight or reducing BMI are provided wherein the method comprises chronically administering an amount of an NMX Peptide, FNX Peptide or NMX Receptor agonist to a subject in need or desirous thereof. In one embodiment, the NMX Peptide, FNX Peptide or NMX Receptor agonist is administered in an extended release, slow release, sustained release or long acting formulation. In one embodiment, the NMX Peptide, FNX Peptide or NMX Receptor agonist is administered in a polymer-based sustained release formulation, such as PLGA polymer based vehicles.

Based upon the pharmacological activities described herein, NMX Peptides, FNX Peptides or NMX Receptor Agonists may be useful for the treatment of metabolic diseases (including various manifestations of diabetes mellitus and dysglycemia, insulin resistance and insulin resistance syndrome, obesity, dyslipidemia).

Additionally, NMX Peptides, FNX Peptides or NMX Receptor Agonists may be used to treat conditions or disorders which can be alleviated by reducing caloric (or nutrient) intake or availability. This would include any condition or disorder in a subject that is either caused by, complicated by, or aggravated by a relatively high nutrient intake or availability, or that can be alleviated by reducing nutrient intake or availability, for example by decreasing food intake. Such conditions or disorders include, but are not limited to, obesity, diabetes mellitus, including type 2 diabetes, eating disorders, and insulin-resistance syndromes.

In one embodiment, the FNX Peptide is administered at times of the day when the subject is most likely to experience the eating disorder. For example, the FNX Peptide is administered at times of the day when the subject is most likely to experience the binge eating. Binge eating can be characterized by 1) eating, in a discrete period of time (e.g., within any 2 hour period), an amount of food that is definitely larger than most people would eat during a similar period of time and under similar circumstances and 2) a sense of lack of control over eating during the episode (e.g., a feeling that one cannot stop eating or control what or how much one is eating). Reducing or inhibiting food intake or suppressing or reducing appetite at these times can reduce the binge eating and the unwanted or deleterious effects of binge eating. In one embodiment, FNX Peptides for use in controlling binge eating include FNX Peptides that have a longer half-life in vivo than native FNX Peptides.

In one embodiment, methods for reducing the deleterious and/or undesirable effects resulting from an eating disorder, such as binge eating, are provided, where the methods comprise administering to a subject in need thereof a FNX Peptide in an amount effective to reduce the eating disorder, e.g. binge eating, by the subject. The reduction of the eating disorder includes a reduction in the frequency of eating disorder episodes, the duration of eating disorder episodes, the total amount consumed during an eating disorder episode, difficulty in resisting the onset of an eating disorder episode, and any combination thereof, as compared to as compared to such frequency, duration, amount and resistance in the absence of administration of a FNX Peptide. These effects are achieved by the reduction or inhibition of food, caloric or nutrient intake or availability or by suppression or reduction of appetite, for example prior to or during an episode of an eating disorder. As such, by way of example, in one embodiment, a method may comprise a reduction in the frequency of binge eating episodes. In another embodiment, a method may comprise a reduction in the duration of binge eating episodes. In yet another embodiment, a method may comprise a reduction in the total amount consumed during a binge-eating episode. In yet another embodiment, a method may comprise a reduction in difficulty resisting the onset of a binge-eating episode.

In some embodiments, the eating disorder, e.g. binge eating, night-eating, is specifically eating of sweet foods, chocolaty foods, savory foods, high fat foods, or any combination thereof, under stressed or non-stressed conditions. In one embodiment, the eating is specifically eating of savory foods, including high fat foods. In one embodiment, the eating is specifically eating of sweet foods, both under stressed and non-stressed conditions.

Eating disorders can typically be determined or measured using a questionnaire or tracking and monitoring daily logs of eating patterns. For example, binge eating can be determined or measured using a questionnaire and a Binge Eating Scale (BES). Binge eating severity can be divided into three categories (mild, moderate, and severe) based on the total BES score (calculated by summing the scores for each individual item). Accordingly, methods are provided for reducing or normalizing the pertinent eating disorder score of a subject comprising administering to a subject in need thereof an FNX Peptide in an amount effective to reduce or normalize the eating disorder score of the subject. In some embodiments, administration of an FNX Peptide changes the category of the subject, for example, from severe to moderate, from severe to mild, or from moderate to mild. For example, methods are provided for reducing the BES score of a subject comprising administering to a subject in need thereof an FNX Peptide in an amount effective to reduce the BES score of the subject. In some embodiments, administration of an FNX Peptide changes the BES category of the subject, for example, from severe to moderate, from severe to mild, or from moderate to mild.

Some of the signs of an eating disorder, e.g. binge eating, night-eating, include eating large amounts of food when not physically hungry, rapid eating, hiding of food because the person feels embarrassed about how much he or she is eating, eating until uncomfortably full, or any combination thereof. In one embodiment, the eating is in response to stressed conditions. Others with eating disorders are substance abusers, such as drug abusers or alcohol abusers. Not everyone who has an eating disorder is overweight, such as those diagnosed with bulimia nervosa.

Subjects who have an eating disorder often over eat or eat inappropriately (e.g. selective for savory and high fat foods) at particular times of the day, and thus treatment should be adjusted according to when the subject is most likely to do so. For example, if the subject binge eats mostly after 7 p.m. at night, the subject should be administered the FNX Peptide at or shortly before 7 p.m. In one embodiment, the subject is administered the FNX Peptide at the time they are susceptible to over eating or inappropriate eating. In other embodiments, the subject is administered the FNX Peptide at least about 15 minutes, at least about 30 minutes, at least about 45 minutes, at least about 1 hour, at least about 1 hour and 30 minutes, or at least about 2 hours before they are susceptible to such eating, e.g. binge eating.

Accordingly, an effective amount of FNX Peptide in such embodiments is an amount effective to curb or control the subject's desire to over eat or eat inappropriately, e.g. binge eat, by reducing or inhibiting food intake or by reducing or suppressing appetite. Therefore, the effective amount of FNX Peptide will change dependent upon the subject and the level of their desire to over eat or eat inappropriately. Furthermore, if a subject's desire to over eat or eat inappropriately is less at one point in the day than at another, the dosage can be adjusted accordingly to provide a lower dose at the times of the day the subject has a lower desire to over eat or eat inappropriately, and to provide a higher dose at the times of the day the subject has a higher desire to over eat or eat inappropriately. In one embodiment, the subject is administered a peak dosage of FNX Peptide at the time they have a high desire to over eat or eat inappropriately. In other embodiments, the subject is administered a peak dosage of FNX Peptide at least about minutes, at least about 30 minutes, at least about 45 minutes, at least about 1 hour, at least about 1 hour and 30 minutes, or at least about 2 hours before they have a high desire to over eat or eat inappropriately.

In one embodiment, the present application provides methods for reducing weight in a subject, where the method comprises the administration of an amount of an NMX Peptide, FNX Peptide or NMX Receptor agonist effective to cause weight reduction to the subject. In another embodiment, the method comprises the chronic or sustained administration of an amount of an NMX Peptide, FNX Peptide or NMX Receptor agonist effective to cause weight reduction to the subject. In still another embodiment, the weight reduction is due to a reduction in body fat or adipose tissue without a corresponding reduction in lean body mass or muscle mass. In still another embodiment, the reduction in body weight due to loss of body fat is greater than the reduction in weight due to loss of lean body mass or muscle mass. In one embodiment the reduction in body fat as compared to lean tissue or muscle is based on an absolute weight basis while in another embodiment it is based a percent of weight lost basis. In yet another embodiment the application provides methods for altering body composition, for example by reducing the ratio of fat to lean tissue, reducing the percent body fat, or increasing the percent lean tissue in an individual.

As used herein, “weight reduction” refers to a decrease in a subject's body weight. While the invention does not depend on any particular reduction in the subject's weight, the methods provided herein will, in various embodiments, reduce the subject's weight by at least about 1%, 2%, 3%, 4%, 5%, 10%, 15, 20%, 30%, 40%, 50%, 60%, or 70% compared to the subject's body weight prior to initiation of the methods disclosed herein. In various embodiments, the weight reduction occurs over a period of about 1 week, 2 weeks, 3, weeks, 1 month, 2 months, 3 months, 4 months, 5 months, 6 months, 7 months, 8 months, 9 months, 10 months, 11 months, 1 year or more. In other embodiments, the subject may lose about 5, 6, 7, 8, 9, 10, 15, 20, 25, 30, 35, 40, 45, 50 100, 125, 150, 175, 200 or more pounds. A reduction in weight can be measured using any reproducible means of measurement. In one embodiment, weight reduction can be measured by calculating a subject's body mass index and comparing that subject's BMI over a period of time. Body mass index can be calculated using any method available, for example by using a nomogram or similar device.

In further embodiments, any of the methods disclosed herein result in the subject's body weight being reduced by at least 1%, at least 5%, at least 10%, at least 20%, at least 30%, at least 40% or at least 50%. In additional embodiments, any of the methods disclosed herein result in the subject's body weight being reduced by at least about 5 pounds or 2 kg, at least about 10 pounds or 5 kg, at least about 20 pounds or 10 kg, at least about 30 pounds or 15 kg, at least about 40 pounds or 20 kg, at least about 50 pounds or 25 kg, at least about 75 pounds or 35 kg, at least about 100 pounds or 50 kg, at least about 125 pounds or 55 kg, at least about 150 pounds or 75 kg, at least about 175 pounds or 80 kg, or at least about 200 pounds or 100 kg. In still further embodiments, practice of any of the methods disclosed herein results in weight reduction, wherein less than about 40%, less than about 20%, less than about 10%, less than about 5%, less than about 2%, less than about 1%, or 0% of the weight loss is due to loss of mean body mass.

The administered NMX Peptide, FNX Peptide or NMX Receptor agonist may be in the form of a peptide, a prodrug, or as a pharmaceutical salt or salts thereof. The term “prodrug” refers to a compound that is a drug precursor that, following administration, releases the drug in vivo via some chemical or physiological process, for example, proteolytic cleavage, or upon reaching an environment of a certain pH.

Methods provided herein can be used on any individual in need of such methods or individuals for whom practice of the methods is desired. These individuals may be any mammal including, but not limited to, humans, dogs, horses, cows, pigs, chicken and other commercially valuable or companion animals.

In the methods of treatment described herein, the novel FNX Peptides may be administered by any means known in the art, peripherally, intestinally, intracerebrally or intracerebrovascularly, and the like. In view of the Applicant's discovery that neuromedins can act peripherally in mammals to reduce food intake, it is to be understood that additional embodiments are expressly intended in which an NMX Peptide or NMX Receptor agonist replaces an FNX Peptide in any method of treatment use described herein, e.g. to reduce or control binge eating or other eating disorder, when the NMX Peptide or NMX Receptor agonist is administered systemically or peripherally and other than intracerebrally or intracerebrovascularly.

NMX Peptides, FNX Peptides or NMX Receptor Agonists may further be used for screening other compounds having a property of the NMX Peptides, FNX Peptides or NMX Receptor Agonists described herein. Exemplary screening methods are described in PCT application WO 2004/048547, the contents of which are incorporated by reference in its entirety. The present invention provides for antibodies specific for an NMX Peptides, FNX Peptides or NMX Receptor Agonists. Moreover, NMX Peptides, FNX Peptides or NMX Receptor Agonists and/or their antibodies can also be used in diagnostic applications for determining, or propensity for developing, conditions or disorders as described herein.

### FNX Peptides

Also provided are novel FNX Peptides. In one embodiment novel FNX Peptides comprise an amino acid sequence of formula (I): F1-P, where F1-P is a novel combination of an F1 and P segments, where P is an octapeptide as described herein capable of providing, when attached to the F1 portion and systemically delivered, suppression of food intake, reduction of body weight, and/or induction of a satiety signal, and wherein F1 is a des-octapeptide portion of an FN38 or analog or derivative or chimera thereof, as described herein, which enhances or enables P activity. Additional octapeptides and F1 portions are disclosed herein, as well as methods to make and identify additional FNX Peptides. Excluded from F1-P are the known FN38 related compounds, GenBank Accession Number AJ510133 (human), CAD52851 (rat), CAD52850 (frog) and chicken FN38, however their respective F and P segments can be used to create the novel FNX Peptides described herein.

In another embodiment novel FNX Peptides comprise an amino acid sequence of formula (II): F2-P, where P is an octapeptide as described herein capable of providing, when attached to the F2 portion and systemically delivered, suppression of food intake, reduction of body weight, and/or induction of a satiety signal, and wherein F2 is a des-octapeptide portion of an FN38 and SN23 chimera or analog or derivative thereof, as described herein, which enhances or enables P activity. An exemplary effective hybrid is FN38(1-15)-SN23 (FLFHYSKTQKLGKSNSDEEVQVPGGVISNGYFLFRPRN-NH2; SEQ ID No.: 2), which is a hybrid of tree frog SN-23 NMU (SDEEVQVPGGVISNGYFLFRPRN-NH2; SEQ ID No.: 3) and human FN38 (FLFHYSKTQKLGKSNVVEELQSPFASQSRGYFLFRPRN-NH2; SEQ ID No.: 4), additionally in its amide form (the “-NH2” indicating a C-terminal amide). In this embodiment the “P” octapeptide is YFLFRPRN (SEQ ID NO. 5) and the “F” portion is FLFHYSKTQKLGKSNSDEEVQVPGGVISNG (SEQ ID NO. 6). Additional octapeptides and F2 portions are disclosed herein, as well as methods to make and identify additional FNX Peptides

These compounds can be designed to have increased chemical (e.g. stability at pH range desired for formulation and or delivery) and/or enzymatic stability, e.g. peptidase stability (i.e. stability to peptidases and proteinases). In one embodiment the FNX Peptide has a BBM stability value (See the Examples) of at least 70% or greater, at least 75% or greater, at least 80% or greater, at least 90% or greater, or at least 95% or greater.

Additionally, as used herein, an “analog” is defined as a molecule having one or more amino acid substitutions, deletions, inversions, or additions compared with a parent peptide such as FN38. The term “agonist” also includes derivatives. A “derivative” is defined as a molecule having the amino acid sequence of a parent peptide or of an analog of the parent peptide, but additionally having a chemical modification of one or more of its amino acid side groups, alpha-carbon atoms, terminal amino group, or terminal carboxylic acid group. A chemical modification includes, but is not limited to, adding chemical moieties, creating new bonds, and removing chemical moieties.

By “NMX Peptide” is meant a neuromedin U, neuromedin S, an FN36, and an FN38 or analogs and derivatives thereof, including FNX Peptides as described herein. The polypeptide may be obtained or derived from any species. Thus, the term includes the human full-length amino acid peptides, and species variations of thereof, including e.g., murine, hamster, chicken, bovine, rat, and dog polypeptides. In this sense the descriptors wild-type, native and unmodified, are used interchangeably.

By “NMX Receptor agonist” is meant any compound, including peptide, peptide-like compounds and small molecules, that elicits similar biological activities as FN38 and act on a known neuromedin U or S receptor, e.g., NMUR1 or NMUR2. Human NMU-25 is an example of an NMU Receptor agonist.

Exemplary NMX Peptides and NMU Receptor agonists include human neuromedin-25 and:

| | SEQ ID | | |
|---|---|---|---|
| Compd # | No. | Description | Sequence |
| 163291 | 5 | Neuromedin U8 | YFLFRPRN-NH2 |
| | | (porcine) | |
| | | (octapeptide) | |
| 163293 | 10 | Neuromedin U | YKVNEYQGPVAPSGGFFLFRPRN-NH2 |
| | | (rat) | |
| 163357 | 11 | Neuromedin U- | GYFLFRPRN-NH2 |
| | | 9(human) | |
| 163452 | 3 | SN-23(tree frog) | SDEEVQVPGGVISNGYFLFRPRN-NH2 |
| 163661 | 4 | FN-38 (SLM14) | FLFHYSKTQKLGKSNVVEELQSPFASQSRGYFLFRP |
| | | (human) | RN-NH2 |
| | 12 | human | FRVDEEFQSPFASQSRGYFLFRPRN-HN2 |
| | | neuromedin U 25 | |

In certain embodiments, the FNX Peptides can have comparable or higher potency in the treatment and/or prevention of the disease and conditions described herein as compared to native FN38 polypeptides, e.g. FN38, and compared to FN15. In other embodiments, the FNX Peptide can have less (e.g., may be 2, 3, 4, or even 5 times less), though still effective, potency in the treatment and/or prevention of the above described conditions, but may possess other desirable characteristics over native FN38 or compared to FN15, e.g. increased stability or solubility, less side effects, combination of biological activities, and/or ease in manufacturing, formulating, or use.

Exemplary compounds include:

| | SEQ ID | | |
|---|---|---|---|
| Compd # | No. | Description | Sequence |
| 165063 | 2 | FN38(1-15)-SN23 | FLFHYSKTQKLGKSNSDEEVQVPGGVISNGY |
| | | | FLFRPRN-NH2 |
| 165061 | 6 | FN38(1-15)-SN23(des- | FLFHYSKTQKLGKSNSDEEVQVPGGVISNG-- |
| | | octapeptide) | ----------------NH2 |
| 165062 | 14 | FN38(des-octapeptide) | FLFHYSKTQKLGKSNVVEELQSPFASQSRG-- |
| | | | ----------------NH2 |
| 165054 | 12 | HNMU25 | FRVDEEFQSPFASQSRGYFLFRPRN-HN2 |

The peptides may or may not be amidated at the C-terminal end.

In one embodiment FNX Peptides have one of the octapeptide sequences (“P”) below. In further embodiments FNX Peptides have two, three, four, five or six of the octapeptide substitutions shown below.

Exemplary analogs of FNX Peptide 163661 having the above octapeptide sequences and the F1 region of FN38 include:

| SEQ | | |
|---|---|---|
| ID | | |
| No.: | Description | Sequence |
| 15 | FN38-Y31F | FLFHYSKTQKLGKSNVVEELQSPFASQSRGFFLFRPRN-NH2 |
| 16 | FN38-F34V | FLFHYSKTQKLGKSNVVEELQSPFASQSRGYFLVRPRN- |
| | | NH2 |
| 17 | FN38-L33F | FLFHYSKTQKLGKSNVVEELQSPFASQSRGYFFFRPRN-NH2 |
| 18 | FN38-R35H | FLFHYSKTQKLGKSNVVEELQSPFASQSRGYFLFHPRN-NH2 |
| 19 | FN38-R37H | FLFHYSKTQKLGKSNVVEELQSPFASQSRGYFLFRPHN-NH2 |
| 20 | FN38-P36 beta | FLFHYSKTQKLGKSNVVEELQSPFASQSRGYFLFR(beta turn |
| | turn mimic | mimic B)RN-NH2 |

In some embodiments a P region octapeptide does not have a histidine substituting for either or both arginines. In some embodiments a P region octapeptide does not have a beta turn mimic substituting for proline.

Further exemplary analogs of Formula II having the above octapeptide sequences and the F2 region of FN38(1-15)-SN23 (Peptide 165063) include:

| SEQ | | |
|---|---|---|
| ID | | |
| No.: | Description | Sequence |
| 21 | FN38(1-15)- | FLFHYSKTQKLGKSNSDEEVQVPGGVISNGFFLFRPRN-NH2 |
| | SN23-Y31F | |
| 22 | FN38(1-15)- | FLFHYSKTQKLGKSNSDEEVQVPGGVISNGYFLVRPRN- |
| | SN23-F34V | NH2 |
| 23 | FN38(1-15)- | FLFHYSKTQKLGKSNSDEEVQVPGGVISNGYFFFRPRN-NH2 |
| | SN23-L33F | |
| 24 | FN38(1-15)- | FLFHYSKTQKLGKSNSDEEVQVPGGVISNGYFLFHPRN- |
| | SN23-R35H | NH2 |
| 25 | FN38(1-15)- | FLFHYSKTQKLGKSNSDEEVQVPGGVISNGYFLFRPHN- |
| | SN23-R37H | NH2 |
| 26 | FN38(1-15)- | FLFHYSKTQKLGKSNSDEEVQVPGGVISNGYFLFR(beta turn |
| | SN23-P36beta | mimic B)RN-NH2 |
| | turn mimic | |

Additional embodiments include FNX Peptides having multiple substitutions or modifications to the octapeptide region to increase its hydrophobicity and/or its positive charge. Exemplary octapeptide sequences applicable to any FNX Peptide include:

| SEQ | | |
|---|---|---|
| ID | | |
| No.: | Description | Sequence |
| 27 | Octapeptide | FFFYHPHN |
| | (Y1F, L3F, F4Y, R5H, R7H) | |
| 28 | Octapeptide | FFFFRPRN |
| | (Y1F, L3F) | |
| 29 | Octapapetide | FFFFKHHN |
| | (Y1F, L3F, R5K, P6H, R7H) | |
| 30 | Octapeptide | FFFFK(beta turn |
| | (Y1F, L3F, R5K, P6beta | mimic)HN |
| | turn mimic, R7H) | |

Accordingly, in one embodiment are octapeptide substitution analogs, which include for example, substitution analogs of Peptide 163661 including:

| | | |
|---|---|---|
| SEQ | | |
| ID | | |
| No.: | Description | Sequence |

| | | |
|---|---|---|
| 31 | FN38 (Y31F, L33F, | FLFHYSKTQKLGKSNVVEELQSPFASQSRG FFFYHPHN |
| | F34Y, R35H, R37H) | |
| 32 | FN38 (Y31F, L33F) | FLFHYSKTQKLGKSNVVEELQSPFASQSRG FFFFRPRN |
| 33 | FN38 (Y31F, L33F, | FLFHYSKTQKLGKSNVVEELQSPFASQSRG FFFFKHHN |
| | R35K, P36H, R37H) | |
| 34 | FN38 (Y31F, L33F, | FLFHYSKTQKLGKSNVVEELQSPFASQSRG FFFFK(beta |
| | R35K, P36turn mimic, | turn mimic)HN |
| | R37H) | |

Further, in one embodiment are octapeptide substitution analogs, which include for example, substitution analogs of Peptide 165063 chimera including:

| SEQ | | |
|---|---|---|
| ID | | |
| No.: | Description | Sequence |
| 35 | FN38(1-15)-SN23 | FLFHYSKTQKLGKSNSDEEVQVPGGVISNGFFFYHPHN |
| | (Y31F, L33F, F34Y, | |
| | R35H, R37H) | |
| 36 | FN38(1-15)-SN23 | FLFHYSKTQKLGKSNSDEEVQVPGGVISNGFFFFRPRN |
| | (Y31F, L33F) | |
| 37 | FN38(1-15)-SN23 | FLFHYSKTQKLGKSNSDEEVQVPGGVISNGFFFFKHHN |
| | (Y31F, L33F, R35K, | |
| | 36H, R37H) | |
| 38 | FN38(1-15)-SN23 | FLFHYSKTQKLGKSNSDEEVQVPGGVISNGFFFFK(turn |
| | (Y31F, L33F, R35K, | mimic)HN |
| | P36beta turn mimic, | |
| | R37H) | |

In one embodiment FNX Peptides have one or more amino acid deletions, for example, the deletions shown below. In another embodiment an FNX Peptide has two such deleted regions. In another embodiment, a FNX Peptide has at least one amino acid deletion, the amino acid being any one of the amino acids contained within any of the deleted regions shown below. In other embodiments, one, two, three, four, or five amino acids are deleted. Accordingly, depending on the length of the parent peptide, the FNX Peptide may be at least or equal to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, or 43 residues in length or any combination thereof, e.g. at least 10 but no more than 15 residues. In one such embodiment the deleted amino acids are one of the amino acids contained in any of the deleted regions shown below. Accordingly, in one embodiment are deletion analogs, which include for example, deletion and/or substitution analogs of Compound 163661:

| SEQ | | |
|---|---|---|
| ID | | |
| No.: | Description | Sequence |
| 39 | FN38(des 16, 17) | FLFHYSKTQKLGKSN--EELQSPFASQSRGYFLFRPRN-NH2 |
| 40 | FN38(des 24-27) | FLFHYSKTQKLGKSNVVEELQSP----SRGYFLFRPRN-NH2 |
| 41 | FN38(des 16, 17)(des | FLFHYSKTQKLGKSN-EELQSP----SRGYFLFRPRN-NH2 |
| | 24-27) | |
| 42 | FN38(des 1-4) | ----YSKTQKLGKSNVVEELQSPFASQSRGYFLFRPRN-NH2 |
| 43 | FN38(des 6-9) | FLFHY----KLGKSNVVEELQSPFASQSRGYFLFRPRN-NH2 |
| 44 | FN38(des 13-19) | FLFHYSKTQKLG-------LQSPFASQSRGYFLFRPRN-NH2- |
| | | NH2 |
| 45 | FN38(des 2-8) | F-------QKLGKSNVVEELQSPFASQSRGYFLFRPRN-NH2 |
| 46 | Des-(Lys7-Pro23)FN38 | FLFHYS ----------------FASQSRGYFLFRPRN-NH2 |
| 47 | Des-(Val16- | FLFHYSKTQKLGKSN ------------GYFLFRPRN-NH2 |
| | Arg29)FN38 | |
| 48 | Des-(Val16- | FLFHYSKTQKLGKSN ----------SRGYFLFRPRN-NH2 |
| | Gln27)FN38 | |
| 49 | Des- | FLFHYSKTQKLGKSN--EELQSP----SRGYFLFKPRN-NH2 |
| | (Val16, Val17, Phe24---- | |
| | Gln27)[K35]FN38 | |
| 50 | Des-(Lys7-Gly30) | FLFHYS-----------------------GYFLFRPRN-NH2 |
| | FN15 | |
| 51 | Des-(Phe1---------------- | ---------KLGKSNVVEELQSPFASQSRGYFLFRPRN-NH2 |
| | --------Gln9)-FN38 | |
| 52 | Des-(Phe1- | ----YSKTQKLGKSN--EELQSP----SRGYFLFRPRN-NH2 |
| | His4, Val16, Val17, Phe2 | |
| | 4-Gln27)-FN38 | |
| 53 | [MimicB13]FN15 | FLFHYS-------------------------GYFLFR(Mimic B)RN-NH2 |
| 54 | [MimicB13, Phe8]]FN1 | FLFHYS-------------------------GFFLFR(Mimic B)HN-NH2 |
| | 5 | |
| 55 | [Lys12]FN15 | FLFHYS-------------------------GYFLFKPRN-NH2 |
| 56 | [Phe8]FN15 | FLFHYS-------------------------GFFLFRPRN-NH2 |
| 57 | [Lys12, Phe8]FN15 | FLFHYS-------------------------GFFLFKPRN-NH2 |

Accordingly, in one embodiment are deletion analogs, which include for example, deletion analogs of Compound 165063 including:

| SEQ | | |
|---|---|---|
| ID | | |
| No.: | Description | Sequence |
| 58 | FN38(1-15)-SN23(des | FLFHYSKTQKLGKSN--EEVQVPGGVISNGYFLFRPRN- |
| | 16, 17) | NH2 |
| 59 | FN38(1-15)-SN23 (des | FLFHYSKTQKLGKSNSDEEVQVP----SNGYFLFRPRN -NH2 |
| | 24-27) | |
| 60 | FN38(1-15)-SN23 (des | FLFHYSKTQKLGKSN--EEVQVP----SNGYFLFRPRN-NH2 |
| | 16, 17)(des 24-27) | |
| 61 | FN38(1-15)-SN23 (des | ----YSKTQKLGKSNSDEEVQVPGGVISNGYFLFRPRN -NH2 |
| | 1-4) | |
| 62 | FN38(1-15)-SN23 (des | FLFHY----KLGKSNSDEEVQVPGGVISNGYFLFRPRN -NH2 |
| | 6-9) | |
| 63 | FN38(1-15)-SN23 (des | FLFHYSKTQKLG-------VQVPGGVISNGYFLFRPRN-NH2 |
| | 13-19) | |
| 64 | FN38(1-15)-SN23 (des | F-------QKLGKSNSDEEVQVPGGVISNGYFLFRPRN-NH2 |
| | 2-8) | |

It has been found that the N-terminal region of FN38, FLFHYS (SEQ ID NO.: 77), is sufficient to provide or enhance stability and activity of a neuromedin or FN38 octapeptide region or its analog or derivative. Accordingly, in further FNX Peptides the F1 region is FLFHYS (SEQ ID NO.: 77) and P is as described herein. An exemplary analog based on the human FN38 sequence is FLFHYSGYFLFRPRN (SEQ ID NO.: 65), which is also referred to herein as FN15 or Des-(Lys7-Gly30)FN38. Further exemplary analogs, such as substitution analogs, include those in the following table

| | | |
|---|---|---|
| 65 | Des-(Lys7-Gly30)- | FLFHYS-----------------------GYFLFRPRN-NH2 |
| | FN38 | |
| 66 | [MimicB13]FN15 | FLFHYS-------------------------GYFLFR(Mimic B)RN-NH2 |
| 67 | [MimicB13, Phe8]]FN | FLFHYS-------------------------GFFLFR(Mimic B)HN-NH2 |
| | 15 | |
| 68 | [Lys12]FN15 | FLFHYS-------------------------GYFLFKPRN-NH2 |
| 69 | [Phe8]FN15 | FLFHYS-------------------------GFFLFRPRN-NH2 |
| 70 | [Lys12, Phe8]FN15 | FLFHYS-------------------------GFFLFKPRN-NH2 |

In yet a further embodiment the FNX Peptide is selected from the group consisting of FN15, [Lys12]FN15, [Phe8]FN15, [Lys12,Phe8]FN15 and their analogs and derivatives, including the amide form. For example, as discussed herein the FN15 analog, as an FNX Peptide, can have improved chemical and/or enzymatic stability compared to FN15 or FN38.

Exemplary peptides herein display inhibition of food intake as well as a further property of stability in a human brush border membrane assay. For example, the following table presents FNX Peptides with greater than 25% food intake inhibition in a mouse assay at a dose of 200 mg/kg measured at 60 minutes. Stability in the human brush border membrane assay as percent compound remaining after a 5 hour incubation is also indicated.

| | Food | |
|---|---|---|
| | Intake | |
| FNX Peptide | Inhibition | hBBM |
| FN38 | −46 | 62 |
| FN38(1-15)-SN23 | −88 | 73 |
| (Phe31]FN38 | −64 | 81 |
| [Val33]FN38 | −51 | 82 |
| [Phe33]FN38 | −32 | 67 |
| Des-Val16,Val17-FN38 | −60 | 73 |
| Des-(Phe24,Ala25,Ser26,Gln27)-FN38 | −83 | 61 |
| Des-(Val16,Val17,Phe24,Ala25,Ser26,Gln27)FN38 | −79 | 77 |
| Des-(Phe1,Leu2,Phe3,His4)FN38 | −58 | 41 |
| Des-(Ser6,Lys7,Thr8,Gln9)FN38 | −72 | 28 |
| Des-(Lys7-Pro23)FN38 | −26 | 95 |
| Des-(Val16-Arg29)FN38 | −42 | 91 |
| Des-(Val16-Gln27)FN38 | −42 | 80 |
| Des-(Val16,Val17,Phe24-Gln27)[K35]FN38 | −54 | 76 |
| Des-(Lys7-Gly30)FN38 or FN15 | −40 | 49 |
| Des-(Phe1-Gln9)FN38 | −70 | 55 |
| Des-(Phe1-His4,Val16,Val17,Phe24-Gln27)FN38 | −69 | 67 |

An FNX Peptide may also include polypeptides having an amino acid sequence with at least 80, 82, 84, 86, 88, 90, 92, 94, 96, 97, or 98% amino acid identity to any FNX Peptide amino acid sequence herein, e.g. FN38 or FN15, and having 1) a similar or superior activity or stability, wherein the FNX Peptide is not a known species variant of FN38, as disclosed herein. Percent identity is determined by analysis with the AlignX® module in Vector NTI® (Invitrogen; Carlsbad Calif.).

In one embodiment an FNX Peptide is one having an amino acid sequence with at least 80, 82, 84, 86, 88, 90, 92, 94, 96, 97, or 98% amino acid identity to FN38 amino or FN15 acid sequence herein and having 1) a similar or superior activity or stability, wherein the FNX Peptide is not a known species variant of FN38 as disclosed herein. Percent identity is determined by analysis with the AlignX® module in Vector NTI® (Invitrogen; Carlsbad Calif.).

In another embodiment an FNX Peptide is one having an amino acid sequence with at least 80, 82, 84, 86, 88, 90, 92, 94, 96, 97, or 98% amino acid identity to FN38(1-15)-SN23 hybrid amino acid sequence herein and having 1) a similar or superior activity or stability, wherein the FNX Peptide. Percent identity is determined by analysis with the AlignX® module in Vector NTI® (Invitrogen; Carlsbad Calif.).

The superior activity may be NMU Receptor binding or activation, reduction of food intake or weight loss or improved chemical or enzymatic stability, e.g. plasma or BBM stability. Stability can be measured in the BBM assay or a plasma assay.

Compounds may further include additional amino acids, chemicals, or moieties that do not affect the biological activity or function of the peptide but may perform other functions, such as aiding purification (e.g., histidine tag), detection (e.g., biotin), increasing solubility or half-life (e.g. pegylation) or expression (e.g., secretion signal peptide).

The FNX Peptides may also be further derivatized by chemical alterations such as amidation, glycosylation, acylation, sulfation, phosphorylation, acetylation, and cyclization. Such chemical alterations may be obtained through chemical or biochemical methodologies, as well as through in vivo processes, or any combination thereof. Derivatives of the analog polypeptides may also include conjugation to one or more polymers or small molecule substituents. One type of polymer conjugation is linkage or attachment of polyethylene glycol (“PEG”) polymers, polyamino acids (e.g., poly-his, poly-arg, poly-lys, etc.) and/or fatty acid chains of various lengths to the N- or C-terminus or amino acid residue side chains of an FNX Peptide. Small molecule substituents include short alkyls and constrained alkyls (e.g., branched, cyclic, fused, adamantyl), and aromatic groups. In addition, basic residues such as R and K may be replaced with homoR and homoK, citrulline, or ornithine to improve metabolic stability of the peptide. FNX Peptides also include acid as well as amide forms of the peptides.

FNX Peptide also include biologically active fragments of the larger peptides described herein. Examples of the desired activity include (1) having activity in a food intake, gastric emptying, pancreatic secretion, blood pressure, heart rate or weight loss assay similar to an FNX Peptide, and/or (2) binding in a receptor binding assay for an NMX Receptor (e.g., NMUR1, NMUR2).

In one embodiment, an FNX Peptide will bind receptor with an affinity of greater than 1 uM, and, in another embodiment, with an affinity of greater than 1-10 nM.

By a polypeptide having “FNX Peptide” is meant that the polypeptide demonstrates similar physiological characteristics as FN38, such as those described in the instant specification, for example, in reducing food intake. The polypeptides of the present invention may be capable of binding to or otherwise directly or indirectly interacting with an NMX Receptor, or other receptor or receptors with which FN38 itself may interact to elicit a biological response, e.g., reducing food intake.

Given the biological activity described herein, the present invention provides FNX Peptide compositions for use in a medicament for treating a disease or disorder in a subject in need thereof. The present invention also provides methods for use of FNX Peptide compositions in treating a disease or disorder in a subject.

By “amino acid” and “amino acid residue” is meant natural amino acids, unnatural amino acids, and modified amino acid, all in their D and L stereoisomers if their structure allows such stereoisomeric forms. Natural amino acids include alanine (Ala), arginine (Arg), asparagine (Asn), aspartic acid (Asp), cysteine (Cys), glutamine (Gln), glutamic acid (Glu), glycine (Gly), histidine (His), isoleucine (Ile), leucine (Leu), Lysine (Lys), methionine (Met), phenylalanine (Phe), proline (Pro), serine (Ser), threonine (Thr), tryptophan (Trp), tyrosine (Tyr) and valine (Val). Unnatural amino acids include, but are not limited to azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homolysine, homoproline, homoserine, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid and thioproline, homolysine, homoarginine, homoserine, citrulline, ornithine, Ne-formyllysine. Modified amino acid include the natural and unnatural amino acids which are chemically blocked, reversibly or irreversibly, or modified on their N-terminal amino group or their side chain groups, as for example, methionine sulfoxide, methionine sulfone, S (carbo amino group or side chain functional group has been chemically codified to another functional group. For example, aspartic acid-(beta-methyl ester) is a modified amino acid of aspartic acid; N-ethylglycine is a modified amino acid of glycine; or alanine carboxamide is a modified amino acid of alanine. Additional residues that can be incorporated are described by Sandberg et. al. (1998) J. Med. Chem. 41:2481-2491.

In one embodiment, the FNX Peptide retains or affects, by at least about 25%, about 30%, 40%, 50%, 60%, 70%, 80%, 90%, 95%, 98%, or 99%, the biological activity of an FN38 polypeptide or another polypeptide of the FN38/36 family or FN15. In another embodiment, the agonist analog polypeptides exhibit improved activity over at least one of the other FN38/36 polypeptides, FN38 or FN15. For example, the agonist analog polypeptides exhibit at least about 110%, 125%, 130%, 140%, 150%, 200%, or more of the biological activity of FN38 polypeptide or another polypeptide of the FN36/38 family, e.g FN38 or FN15. An exemplary function of FN38 and FN15 is the reduction of food intake or reduction of body weight.

Exemplary FNX Peptides are those having a potency in one of the assays described herein (for example, receptor binding assays, food intake, and/or weight reduction assays) which is greater than or equal to the potency of human FN38 polypeptide or FN15 in that same assay. For example, the FNX Peptides may bind to at least one of the receptors with an affinity of greater than 30 nM, 20 nM, 10 nM, or more. However, it is also contemplated that FNX Peptides can have less potency in the assays. FNX Peptides may further possess desirable characteristics, such as a specific binding profile, stability, solubility, or ease in manufacturing or formulation.

In one example, the polypeptides of the present invention may demonstrate activity in food intake assays. Such polypeptides demonstrate the ability to reduce cumulative food intake by more than 5% over administration of the vehicle, more than 15%, more than 25%, more than 35%, or more than 50% over the vehicle. In a one embodiment, the FNX Peptide reduces food intake by more than 75 or even 90%.

In another general aspect, the invention includes nucleic acids that can encode the FNX Peptides herein described. Such nucleic acids can be determined from the amino acid sequences provided herein using standard coding table well known in the art.

In one embodiment the FNX Peptide by proviso specifically excludes Compound A, excludes Compound B, excludes Compound C, and/or excludes Compound D. In one embodiment NMX Receptor agonists specifically excludes rutin and its analogs and derivatives that bind an NMU receptor. In another embodiment NMX Receptor agonists specifically excludes non-peptides that may bind an NMU receptor, e.g. rutin. In yet other embodiments the FNX peptide octapeptide region P optionally does not have a histidine for arginine substitution, and optionally, does not have a beta turn mimic substituting for proline.

### Making NMX Peptides, FNX Peptides and NMX Receptor Agonists

The compounds described herein may be prepared using standard recombinant techniques or chemical peptide synthesis techniques known in the art, e.g., using an automated or semi-automated peptide synthesizer, or both. Likewise, the derivatives of the polypeptides may be produced using standard chemical, biochemical, or in vivo methodologies.

The compounds can be synthesized in solution or on a solid support in accordance with conventional techniques. Various automatic synthesizers are commercially available and can be used in accordance with known protocols. See, e.g., Stewart and Young, Solid Phase Peptide Synthesis, 2d. ed., Pierce Chemical Co. (1984); Tam et al, J. Am. Chem. Soc. 105: 6442 (1983); Merrifield, Science 232: 341-7 (1986); and Barany and Merrifield, The Peptides, Gross and Meienhofer, eds., Academic Press, New York, 1-284 (1979).

The compounds may alternatively be produced by recombinant techniques well known in the art. See, e.g., Sambrook et al, Molecular Cloning: A Laboratory Manual, 2d ed., Cold Spring Harbor (1989). These polypeptides produced by recombinant technologies may be expressed from a polynucleotide, e.g., a DNA or RNA molecule. These polynucleotide sequences may incorporate codons facilitating transcription and translation of mRNA in host cells. Such manufacturing sequences may readily be constructed according to the methods well known in the art. See, e.g., WO 83/04053. A variety of expression vector/host systems may be utilized to contain and express a compound-coding sequence.

As such, the amino acid sequences of the compounds determine the polynucleotide sequences that are useful in generating new and useful viral and plasmid DNA vectors, new and useful transformed and transfected prokaryotic and eukaryotic host cells (including bacterial, yeast, algae, plant, insect, avian, and mammalian cells grown in culture), and new and useful methods for cultured growth of such host cells capable of expression of the present polypeptides. The polynucleotide sequences encoding the compounds can also be used for gene therapy.

DNA sequences encoding the compounds may be created using well known molecular biology (or recombinant) techniques such as amplification by PCR or site directed mutagenesis and cloning into an appropriate vector, for example, pGEX-3X (Pharmacia, Piscataway, N.J.).

The present invention also provides for processes for the production of the present compounds (NMX peptides, FNX peptides or NMX Receptor agonists). Provided is a process for producing the polypeptides from a host cell containing nucleic acids encoding such compounds comprising: (a) culturing said host cell containing polynucleotides encoding a compound under conditions facilitating the expression of such DNA molecules; and (b) obtaining such compounds. Host cells may be prokaryotic or eukaryotic, such as bacterial, yeast, algae, plant, insect, avian, and mammalian cells. Mammalian host cells include, for example, human cells cultured in vitro. Also contemplated are processes of producing polypeptides using a cell free system. An example of a cell free protein expression system is the Rapid Translation System (RTS) by Roche Diagnostics Corp.

A variety of expression vector/host systems may be utilized to contain and express a compound-coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, tobacco mosaic virus) or transformed with bacterial expression vectors (e.g., Ti or pBR322 plasmid); or mammalian cell systems. Mammalian cells that are useful in recombinant protein productions include, but are not limited to, VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Exemplary protocols for the recombinant expression of the protein in any of these expression/host systems, as well as other expression/host systems, are well known in the art.

It is generally desirable to purify the compound. Peptide purification techniques are well known to those of skill in the art. These techniques may involve the crude fractionation of the cellular milieu to polypeptide and non-polypeptide fractions. The polypeptides of interest may be further purified using chromatographic and electrophoretic techniques to achieve partial or complete purification (or purification to homogeneity). Analytical methods particularly suited to the preparation of a pure peptide are ion-exchange chromatography, exclusion chromatography, polyacrylamide gel electrophoresis, and isoelectric focusing. A particularly efficient method of purifying peptides is reverse phase HPLC, followed by characterization of purified product by liquid chromatography/mass spectrometry (LC/MS) and Matrix-Assisted Laser Desorption Ionization (MALDI) mass spectrometry. Additional confirmation of purity is obtained by determining amino acid analysis.

The term “purified peptide” as used herein, is intended to refer to a composition, isolatable from other components, wherein the peptide is purified to any degree relative to its naturally obtainable state. A purified peptide therefore refers to a peptide, free from the environment in which it may naturally occur. The term “substantially purified” is used to refer to a composition in which the peptide forms the major component of the composition, such as constituting at least about 50%, about 60%, about 70%, about 80%, about 90%, about 95% or more of the peptides in the composition. Methods for purifying a polypeptide can be found, for example, in U.S. Pat. No. 5,849,883, incorporated by reference in its entirety.

Various techniques suitable for use in peptide purification are well known in the art. These include, for example, precipitation with ammonium sulfate, PEG, antibodies, and the like; heat denaturation, followed by centrifugation; chromatography steps such as ion exchange, gel filtration, reverse phase, hydroxlyapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the peptides always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed, utilizing an HPLC apparatus, will generally result in a greater “-fold” purification than the same technique utilizing a low pressure chromatography system. Methods exhibiting a lower degree of relative purification may have advantages in total recovery of protein product, or in maintaining the activity of an expressed protein. Also it is contemplated that a combination of anion exchange and immunoaffinity chromatography may be employed to produce purified peptide compositions of the present invention.

### Pharmaceutical Compositions

The present invention also relates to pharmaceutical compositions comprising a therapeutically or prophylactically effective amount of at least an NMX Peptide, FNX Peptide or NMX Receptor agonist, or a pharmaceutically acceptable salt thereof, together with pharmaceutically acceptable diluents, preservatives, solubilizers, emulsifiers, adjuvants and/or carriers useful in the delivery of an NMX Peptide, FNX Peptide or NMX Receptor agonist. Such compositions may include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimersol, benzyl alcohol), and bulking substances (e.g., lactose, mannitol); incorporation of the material into particulate preparations of polymeric compounds, such as polylactic acid, polyglycolic acid, etc., or in association with liposomes. Such compositions will influence the physical state, stability, rate of in vivo release, and rate of in vivo clearance of the compound. See, e.g., Remington's Pharmaceutical Sciences 1435-712, 18th ed., Mack Publishing Co., Easton, Pa. (1990). Exemplary methods for formulating pharmaceutical compositions can be found in WO 2004/048547, the entire contents of which are incorporated by reference.

As used herein, the phrase “pharmaceutically acceptable” refers to an agent that does not interfere with the effectiveness of the biological activity of an active ingredient, and which may be approved by a regulatory agency of the Federal government or a state government, or is listed in the U.S. Pharmacopeia or other generally recognized pharmacopeia for use in animals, and more particularly for use in humans. Accordingly, suitable pharmaceutically acceptable carriers include agents that do not interfere with the effectiveness of a pharmaceutical composition or produce an adverse, allergic or other untoward reaction when administered to an animal or a human.

As used herein, the phrase “pharmaceutically acceptable salts” refers to salts prepared from pharmaceutically acceptable, additionally nontoxic, acids and bases, including inorganic and organic acids and bases, including but not limited to, sulfuric, citric, maleic, acetic, oxalic, hydrochloride, hydro bromide, hydro iodide, nitrate, sulfate, bisulfite, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Pharmaceutically acceptable salts include those formed with free amino groups such as, but not limited to, those derived from hydrochloric, phosphoric, acetic, oxalic, and tartaric acids. Pharmaceutically acceptable salts also include those formed with free carboxyl groups such as, but not limited to, those derived from sodium, potassium, ammonium, sodium lithium, calcium, ferric hydroxides, isopropylamine, triethylamine, 2-ethylamino ethanol, histidine, and procaine.

An NMX Peptide, FNX Peptide or NMX Receptor agonist may be formulated for peripheral (systemic) administration, including formulation for injection, oral administration, nasal administration, pulmonary administration, topical administration, or other types of administration as one skilled in the art will recognize. Additionally, administration of the pharmaceutical compositions according to the present invention may be via any common route so long as the target tissue is available via that route. In an additional embodiment, the pharmaceutical compositions may be introduced into the subject by any conventional peripheral method, e.g., by intravenous, intradermal, intramusclar, intramammary, intraperitoneal, intrathecal, retrobulbar, intrapulmonary (e.g., term release); by oral, sublingual, nasal, anal, vaginal, or transdermal delivery, or by surgical implantation at a particular site. Examples include, intravenous or subcutaneous injection; nasal, oral or muscosal administration; and pulmonary inhalation by nose or mouth. The treatment may consist of a single dose or a plurality of doses over a period of time. Controlled continual release of the compositions of the present invention is also contemplated.

The pharmaceutical compositions suitable for injectable use include sterile aqueous solutions or dispersions and sterile powders for the extemporaneous preparation of sterile injectable solutions or dispersions. In all cases, the form should be sterile and should be fluid to the extent that is easily syringable. It is also desirable for the polypeptide to be stable under the conditions of manufacture and storage and must be preserved against the contaminating action of microorganisms, such as bacteria and fungi. The carrier can be a solvent or dispersion medium containing, for example, water, ethanol, polyol (e.g., sorbitol, glycerol, propylene glycol, and liquid polyethylene glycol, and the like), dimethylacetamide, cremorphor EL, suitable mixtures thereof, and oils (e.g., soybean, sesame, castor, cottonseed, ethyl oleate, isopropyl myristate, glycofurol, corn). The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, meta-cresol, benzyl alcohol, parabens (methyl, propyl, butyl), chlorobutanol, phenol, phenylmercuric salts (acetate, borate, nitrate), sorbic acid, thimerosal, and the like. In many cases, tonicity agents (for example, sugars, sodium chloride) will be included in the compositions. Prolonged absorption of the injectable compositions can be brought about by the use in the compositions of agents delaying absorption (for example, aluminum monostearate and gelatin).

It is further contemplated that these compounds may be delivered by inhalation. The peptides may follow the air-flow to the alveoli. Such delivery of the peptides may include delivery as low or ultra-low density particles, such as “whiffle balls,” for example US2004-0170568 and U.S. Pat. No. 6,630,169 (incorporated herein by reference in their entirety) or TECHNOSPHERES™ (Pharmaceutical Discovery Corporation, Elmsford, N.Y.

In one embodiment, the pharmaceutical compositions of the present invention are formulated so as to be suitable for parenteral administration, e.g., via injection or infusion. In one embodiment, the compound is suspended in an aqueous carrier, for example, in an isotonic buffer solution at a pH of about 3.0 to about 8.0, in another embodiment at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. A form of repository or “depot” slow release preparation may be used so that therapeutically effective amounts of the preparation are delivered into the bloodstream over many hours or days following transdermal injection or delivery.

Generally a therapeutically or prophylactically effective amount of the NMX Peptide, FNX Peptide or NMX Receptor agonist will be determined by the age, weight, and condition or severity of the diseases or metabolic conditions or disorders of the recipient. See, e.g., Remington's Pharmaceutical Sciences 697-773. See also Wang and Hanson, Parenteral Formulations of Proteins and Peptides: Stability and Stabilizers, Journal of Parenteral Science and Technology, Technical Report No. 10, Supp. 42:2 S (1988). Typically, a dosage of between about 0.001 ug/kg body weight to about 1000 ug/kg body weight, may be used, but more or less, as a skilled practitioner will recognize, may be used. Dosing may be one or more times daily, or less frequently, and may be in conjunction with other compositions as described herein. It should be noted that the present invention is not limited to the dosages recited herein.

Appropriate dosages may be ascertained through the use of established assays for determining level of metabolic conditions or disorders in conjunction with relevant dose-response data. The final dosage regimen will be determined by the attending physician, considering factors that modify the action of drugs, e.g., the drug's specific activity, severity of the damage and the responsiveness of the patient, the age, condition, body weight, sex and diet of the patient, the severity of any infection, time of administration and other clinical factors.

An effective dose will typically be in the range of about 1 to 30 ug to about 5 mg/day, alternatively about 10 to 30 ug to about 2 mg/day and in additional embodiments from about 5 to 100 ug to about 1 mg/day, or about 5 ug to about 500 ug/day, for a 50 kg patient, administered in a single or divided doses or controlled continued release. Exemplary dosages are between about 0.01 to about 100 ug/kg/dose. Administration should begin whenever the suppression of nutrient availability, food intake, weight, blood glucose or plasma lipid lowering or blood pressure lowering or increasing is desired, for example, at the first sign of symptoms or shortly after diagnosis of obesity, diabetes mellitus, insulin-resistance syndrome, hypertension or hypotension. Administration may be by any route, e.g., injection (including subcutaneous or intramuscular), oral, nasal, transdermal, etc. Dosages for certain routes, for example oral administration, may be increased to account for decreased bioavailability, for example, by about 5-100 fold.

In one embodiment where the pharmaceutical formulation is to be administered parenterally, the composition may be formulated so as to deliver a dose of an NMX Peptide, FNX Peptide or NMX Receptor agonist ranging from 0.01 ug/kg to 100 mg/kg body weight/day, or at a range of about 0.01 ug/kg to about 500 ug/kg per dose, about 0.05 ug/kg to about 250 ug/kg or below about 50 ug/kg. Another exemplary dose range is from 0.1 mg/kg to about 50 mg/kg body weight/day. Another exemplary dose range is from 0.1 mg/kg to about 50 mg/kg body weight/day. Dosages in these ranges will vary with the potency of each analog or derivative, of course, and may be determined by one of skill in the art. Exemplary body weights contemplated in for the dosing regimen can be about 40, 50, 60, 70, 80, 90, or 100 kg or more. Parenteral administration may be carried out with an initial bolus followed by continuous infusion to maintain therapeutic circulating levels of drug product. Those of ordinary skill in the art can readily optimize effective dosages and administration regimens as determined by good medical practice and the clinical condition of the individual patient.

In one embodiment, the NMX Peptide, FNX Peptide or NMX Receptor agonist is co-administered with at least one other obesity-reducing compound. Such a drug can have this effect by any of a number of means, including, but not limited to, suppressing hunger, controlling appetite, increasing metabolism, etc. The at least one other drug may cause weight loss. The at least one other drug can be administered as a bolus dose or as a continuous dose. By “co-administered” is meant that the NMX Peptide, FNX Peptide or NMX Receptor is administered as a single administration with a second obesity reducing compound, simultaneously as separate doses, or as sequentially administered where the administration of the compounds may be separated in time by seconds, minutes, or hours. Sequential administration refers to administering the NMX Peptide, FNX Peptide or NMX Receptor either before or after the second obesity reducing drug. In an additional aspect, the NMX Peptide, FNX Peptide or NMX Receptor is administered 30 minutes before or after the second obesity drug, and further it can be administered 1, 2, 3, 4, 5, 6, 7, 8, 9, 10, 11, or 12 hours before or after the second obesity drug.

Thus in the methods of the present invention, the polypeptides may be administered separately or together with one or more other compounds and compositions that exhibit a long term or short-term action or complementary action, i.e., combination or adjunct therapy. For example, an additional compound may be added to an NMX Peptide, FNX Peptide or NMX Receptor agonist that also reduces nutrient availability, such compounds include, but are not limited to an amylin or amylin analog agonist, salmon calcitonin, a cholecystokinin (CCK) or CCK agonist, a leptin (OB protein) or leptin agonist, an exendin or exendin agonist or analog agonist, or a GLP-1 or GLP-1 agonist or analog agonist or a PYY or PYY agonist or analog, or a PYY related polypeptide. Suitable amylin agonists include, for example, [25,28,29Pro]-human amylin (also known as “pramlintide,” and described in U.S. Pat. Nos. 5,686,511 and 5,998,367). The CCK used is, for example, CCK octapeptide (CCK-8). Leptin is discussed, for example, in Pelleymounter et al. (1995) Science 269:540-543, Halaas et al. (1995) Science 269:543-546, and Campfield et al. (1995) Science 269:546-549. Suitable exendins include exendin-3 and exendin-4, and exendin agonist compounds including, for example, those described in PCT Publications WO 99/07404, WO 99/25727, and WO 99/25728. Suitable PYY polypeptides and analogs include those described in U.S. Application Nos. 60/543,406 and 60/543,407, PCT publications WO 03/026591 and WO 03/057235. Additional obesity-reducing compounds and diet aids include sibutramine, orlistat, leptin, amylin agonists and rimonabant.

According to the methods provided herein, when co-administered with at least one other obesity reducing (or anti-obesity) or weight reducing drug, a NMX Peptide, FNX Peptide or NMX Receptor agonist Peptide may be: (1) co-formulated and administered or delivered simultaneously in a combined formulation; (2) delivered by alternation or in parallel as separate formulations; or (3) by any other combination therapy regimen known in the art. When delivered in alternation therapy, the methods provided may comprise administering or delivering the active ingredients sequentially, e.g., in separate solution, emulsion, suspension, tablets, pills or capsules, or by different injections in separate syringes. In general, during alternation therapy, an effective dosage of each active ingredient is administered sequentially, i.e., serially, whereas in simultaneous therapy, effective dosages of two or more active ingredients are administered together. Various sequences of intermittent combination therapy may also be used.

As such, in one aspect, the NMX Peptide, FNX Peptide or NMX Receptor agonist Peptides may be used as part of a combination therapy for the control, prevention or treatment of obesity or eating disorders or conditions. Compounds used as part of a combination therapy to control eating, treat obesity or reduce weight include, but are not limited to, central nervous system agents that affect neurotransmitters or neural ion channels, including antidepressants (bupropion), noradrenalin reuptake inhibitors (GW320659), selective serotonin 2c receptor agonists, selective 5HT 2c receptor agonists, antiseizure agents (topiramate, zonisamide), some dopamine antagonists, and caimabinoid-1 receptor antagonists (CB-1 receptor antagonists) (rimonabant); leptin/insulin/central nervous system pathway agents, including leptin analogs, leptin transport and/or leptin receptor promoters, ciliary neurotrophic factor (Axokine), neuropeptide Y and agouti-related peptide antagonists, pro-opiomelanocortin and cocaine and amphetamine regulated transcript promoters, alpha-melanocyte-stimulating hormone analogs, melanocoritin-4 receptor agonists, and agents that affect insulin metabolism/activity, which include protein-tyrosine phosphatase-1 beta inhibitors, peroxisome proliferator activated receptor-7 receptor antagonists, short-acting bromocriptine (ergoset), somatostatin agonists (octreotide), and adiponectin/Acrp3O (Famoxin or Fatty Acid Metabolic Oxidation Inducer); gastrointestinal-neural pathway agents, including those that increase cholecystokinin activity (CCK), PYY activity, NPY activity, and PP activity, increase glucagon-like peptide-1 activity (exendin 4, liraglutide, dipeptidyl peptidase W inhibitors), and those that decrease ghrelin activity, as well as amylin analogs (pramlintide); agents that may increase resting metabolic rate (selective I-3 stimulators/agonist, uncoupling protein homologues, and thyroid receptor agonists); other more diverse agents, including melanin concentrating hormone antagonists, phytostanol analogs, functional oils, P57, amylase inhibitors, growth hormone fragments, synthetic analogs of dehydroepiandrosterone sulfate, antagonists of adipocyte 11 beta-hydroxysteroid dehydrogenase type 1 activity, corticotropin-releasing hormone agonists, inhibitors of fatty acid synthesis (cerulenin and C75), carboxypeptidase, inhibitors, indanone/indanols, aminosterols (trodusquemine/trodulamine), and other gastrointestinal lipase inhibitors (ATL962); amphetamines, such as dextroamphetamine; other sympathomimetic adrenergic agents, including phentermine, benzphetamine, phendimetrazine, mazindol, and diethylpropion.

Other compounds include ecopipam; oxyntomodulin (OM); inhibitors of glucose-dependent insulinotropic polypeptide (GIP); gastrin-releasing peptide; neuromedin B; enterostatin; amfebutamone, SR-586 11; CP-045598; AOD-0604; QC-BT1 6; rGLP-1; 1426 (HhlVIR-1426); N-5984; ISIS-i 13715; solabegron; SR-147778; Org-34517; melanotan-I1; cetiuistat; c-2735; c-5093; c-2624; APD-356; radafaxine; fluasterone; GP-389255; 856464; S-2367; AVE-1625; T-71; oleoyl-estrone; peptide YY(3-36) intranasal; androgen receptor agonists; PYY 3-3 6; DOV-102677; tagatose; SLV-3 19; 1954 (Aventis Pharma AG); oxyntomodulin, Thiakis; bromocriptine, PLIVA; diabetes/hyperlipidemia therapy, Yissum; CKD-502; thyroid receptor beta agonists; beta-3 adrenoceptor agonist; CDK-A agonists; galanin antagonist; dopamine D1/D2 agonists; melanocortin modulators; verongamine; neuropeptide Y antagonists; melanin-concentrating hormone receptor antagonists; dual PPAR alpha/gamma agonists; CGEN-P-4; kinase inhibitors; human MCH receptor antagonists; GHS-R antagonists; ghrelin receptor agonists; DG7O inhibitors; cotinine; CRF-BP inhibitors; urocortin agonists; UCL-2000; impentamine; 3-3 adrenergic receptor; pentapeptide MC4 agonists; trodusquemine; GT-20 16; C-75; CPOP; MCH-1 receptor antagonists; RED-i 03004; aminosterols; orexin-1 antagonists; neuropeptide Y5 receptor antagonists; DRF-4158; PT-i 5; PTPase inhibitors; A372 15; SA-0204; glycolipid metabolites; MC-4 agonist; produlestan; PTP-1 B inhibitors; GT-2394; neuropeptide Y5 antagonists; melanocortin receptor modulators; MLN-4760; PPAR gamma/delta dual agonists; NPY5RA-972; 5-HT2C receptor agonist; neuropeptide Y5 receptor antagonists (jhenyl urea analogs); AGRPIMC4 antagonists; neuropeptide Y5 antagonists (benzimidazole); glucocorticoid antagonists; MCHR1 antagonists; Acetyl-CoA carboxylase inhibitors; R-1496; HOB 1 modulators; NOX-B 11; peptide YY 3-36 (eligen); 5-HT 1 modulators; pancreatic lipase inhibitors; GRC-1087; CB-1 antagonists; MCH-1 antagonists; LY-448 100; bombesin BRS3 agonists; ghrelin antagonists; MC4 antagonists; stearoyl-CoA desaturase modulators; H3 histamine antagonists; PPARpan agonists; EP-0 1492; hormone-sensitive lipase inhibitors; fatty acid-binding protein 4 inhibitors; thiolactone derivatives; protein tyrosine phosphatase 1B inhibitors; MCH-1 antagonist; P-64; PPAR gamma ligands; melanin concentrating hormone antagonists; thiazole gastroprokinetics; PA-452; T-226296; A-33 1440; immunodrug vaccines; diabetes/obesity therapeutics (Bioagency, Biofrontera Discovery GmbH); P-7 (Genfit); DT-0 11 M; PTP 1 B inhibitor; anti-diabetic peptide conjugates; KATP agonists; obesity therapeutics (Lexicon); 5-11T2 agonists; MCH-1 receptor antagonists; GMAD-1/GMAD-2; STG-a-MD; neuropeptide Y antagonist; angiogenesis inhibitors; G protein-coupled receptor agonists; nicotinic therapeutics (ChemGenex); anti-obesity agents (Abbott); neuropeptide Y modulators; melanin concentrating hormone; GW-594884A; MC-4R agonist; histamine 113 antagonists; orphan GPCR modulators; MITO-3 108; NLC-002; HE-2300; IGF/IBP-2-13; 5-HT2C agonists; ML-22952; neuropeptide Y receptor antagonists; AZ-40 140; anti-obesity therapy (Nisshin Flour); GNTI; melanocortin receptor modulators; alpha-amylase inhibitors; neuropeptide Y1 antagonist; beta-3 adrenoceptor agonists; ob gene products (Eli Lilly & Co.); SWR-0342-SA; beta-3 adrenoceptor agonist; SWR-0335; SP-1 8904; oral insulin mimetics; beta 3 adrenoceptor agonists; NPY-1 antagonists; I-3 agonists; obesity therapeutics (7TM Pharma); 11-beta-hydroxysteroid dehydrogenase (HSD)1 inhibitors; QRX-43 1; E-6776; RI-450; melanocortin-4 antagonists; melanocortin 4 receptor agonists; obesity therapeutics (CuraGen); leptin mimetics; A-74498; second-generation leptin; NBI-103; CL-3 14698; CP-1 14271; beta-3 adrenoceptor agonists; N]V11-8739; UCL-1283; BMS-192548; CP-94253; PD-160170; nicotinic agonist; LG-100754; SB-226552; LY-355124; CKD-7 11; L-75 1250; PPAR inhibitors; G-protein therapeutics; obesity therapy (Amylin Pharmaceuticals Inc.); BW-1229; monoclonal antibody (ObeSys/CAT); L-74279 1; (S)-sibutramine; MBU-23; YM-268; BTS-78050; tubby-like protein genes; genomics (eating disorders; Allelix/Lilly); MS-706; GI-264879A; GW-409890; FR-79620 analogs; obesity therapy (Hybrigenics SA); ICI-198157; ESP-A; 5-HT2C agonists; PD-170292; AIT-202; LG-100641; GI-181771; anti-obesity therapeutics (Genzyme); leptin modulator; GHRH mimetics; obesity therapy (Yamanouchi Pharmaceutical Co. Ltd.); SB-25 1023; CP-33 1684; BIBO-3304; cholesten-3-ones; LY-3 62884; BRL-48962; NPY-1 antagonists; A-71 378; ®-didesmethylsibutramine; amide derivatives; obesity therapeutics (Bristol-Myers Squibb Co.); obesity therapeutics (Ligand Pharmaceuticals Inc.); LY-226936; NPY antagonists; CCK-A agonists; FPL-14294; PD-145942; ZA-7114; CL-316243; SR-58878; R-1065; BIBP-3226; HP-228; talibegron; FR-165914; AZM-008; AZM-016; AZM-120; AZM-090; vomeropherin; BMS-187257; D-3800; AZM-131; gene discovery (AxyslGlaxo); BRL-26830A; SX-0 13; ERR modulators; adipsin; AC-253; A-7 1623; A-68552; BMS-210285; TAK-677; MPV-1743; obesity therapeutics (Modex); GI-248573; AZM-134; AZM-127; AZM-083; AZM-132; AZM-1 15; exopipam; SSR-125180; obesity therapeutics (Melacure Therapeutics AB); BRL-35 135; SR-1461 31; P-57; AZM-140; CGP-7 1 583A; RF-105 1; BMS-1 96085; manifaxine; beta-3 agonists; DMNJ (Korea Research Institute of Bioscience and Biotechnology); BVT-5 182; LY-255582; SNX-024; galanin antagonists; neurokinin-3 antagonists; dexfenfluramine; mazindol; diethylpropion; phendimetrazine; benzphetamine; amfebutmone; sertraiine; metforinin; AOD-9604; ATL-062; BVT-933; GT389-255; 5LV319; HE-2500; PEG-axokine; L-796568; and ABT-239.

In some embodiments, compounds for use in combination with a NMX Peptide, FNX Peptide or NMX Receptor agonist Peptide include rimonabant, sibutramine, orlistat, PYY or an analog thereof, CB-i antagonist, leptin, phentermine, and exendin analogs. Exemplary dosing ranges include phentermine resin (30 mg in the morning), fenfluramine hydrochloride (20 mg three times a day), and a combination of phentermine resin (15 mg in the morning) and fenfluramine hydrochloride (30 mg before the evening meal), and sibutramine (10-20 mg). Weinfraub et al. (1984) Arch. Intern. Med. 144:1143-1148.

It will be appreciated that the pharmaceutical compositions and treatment methods may be useful in fields of human medicine and veterinary medicine. Thus, the subject to be treated may be a mammal, for example a human or other animals. For veterinary purposes, subjects include for example, farm animals including cows, sheep, pigs, horses and goats, companion animals such as dogs and cats, exotic and/or zoo animals, laboratory animals including mice, rats, rabbits, guinea pigs and hamsters; and poultry such as chickens, turkeys, ducks and geese.

In addition, the present invention contemplates a kit comprising an NMX Peptide, FNX Peptide or NMX Receptor agonist, components suitable for preparing said compounds for pharmaceutical application, instructions for using said compounds and components for pharmaceutical application.

### REFERENCES

Abiko, T. and Takamura, Y. (2002) Synthesis of two neuromedin U (NMU) analogues and their comparative effect of reducing food intake in rats. Prep Biochem Biotechnol 32, 79-86.
Abiko, T. and Takamura, Y. (2003) Syntheses of two neuromedin U (NMU) analogues and their comparative reducing food intake effect in rats. Amino Acids 25, 107-10.
Augood, S. J., Keast, J. R. and Emson, P. C. (1988a) Distribution and characterisation of neuromedin U-like immunoreactivity in rat brain and intestine and in guinea pig intestine. Regul Pept 20, 281-92.
Austin, C., Lo, G., Nandha, K. A., Meleagros, L. and Bloom, S. R. (1995) Cloning and characterization of the cDNA encoding the human neuromedin U (NmU) precursor: NmU expression in the human gastrointestinal tract. J Mol Endocrinol 14, 157-69.
Austin, C., Oka, M., Nandha, K. A., Legon, S., Khandan-Nia, N., Lo, G. and Bloom, S. R. (1994) Distribution and developmental pattern of neuromedin U expression in the rat gastrointestinal tract. J Mol Endocrinol 12, 257-63.
Ballesta, J., Carlei, F., Bishop, A. E., Steel, J. H., Gibson, S. J., Fahey, M., Hennessey, R., Domin, J., Bloom, S. R. and Polak, J. M. (1988) Occurrence and developmental pattern of neuromedin U-immunoreactive nerves in the gastrointestinal tract and brain of the rat. Neuroscience 25, 797-816.
Benito-Orfila, M. A., Domin, J., Nandha, K. A. and Bloom, S. R. (1991) The motor effect of neuromedin U on rat stomach in vitro. Eur J Pharmacol 193, 329-33.
Bhattacharyya, S., Luan, J., Farooqi, I. S., Keogh, J., Montague, C., Brennand, J., Jorde, L., Wareham, N. J. and O'Rahilly, S. (2004) Studies of the neuromedin U-2 receptor gene in human obesity: evidence for the existence of two ancestral forms of the receptor. J Endocrinol 183, 115-20.
Brighton, P. J., Szekeres, P. G. and Willars, G. B. (2004) Neuromedin U and its receptors: structure, function, and physiological roles. Pharmacol Rev 56, 231-48.
Brown, D. R. and Quito, F. L. (1988) Neuromedin U octapeptide alters ion transport in porcine jejunum. Eur J Pharmacol 155, 159-62.
Cao, C. Q., Yu, X. H., Dray, A., Filosa, A. and Perkins, M. N. (2003) A pro-nociceptive role of neuromedin U in adult mice. Pain 104, 609-16.
Chu, C., Jin, Q., Kunitake, T., Kato, K., Nabekura, T., Nakazato, M., Kangawa, K. and Kannan, H. (2002) Cardiovascular actions of central neuromedin U in conscious rats. Regul Pept 105, 29-34.
Cimini, V., Van Noorden, S., Timson, C. M. and Polak, J. M. (1993) Modulation of galanin and neuromedin U-like immunoreactivity in rat corticotropes after alteration of endocrine status. Cell Tissue Res 272, 137-46.
Conlon, J. M., Domin, J., Thim, L., DiMarzo, V., Morris, H. R. and Bloom, S. R. (1988) Primary structure of neuromedin U from the rat. J Neurochem 51, 988-91.
Domin, J., Al-Madani, A. M., Desperbasques, M., Bishop, A. E., Polak, J. M. and Bloom, S. R. (1990) Neuromedin U-like immunoreactivity in the thyroid gland of the rat. Cell Tissue Res 260, 131-5.
Domin, J., Ghatei, M. A., Chohan, P. and Bloom, S. R. (1986) Characterization of neuromedin U like immunoreactivity in rat, porcine, guinea-pig and human tissue extracts using a specific radioimmunoassay. Biochem Biophys Res Commun 140, 1127-34.
Domin, J., Ghatei, M. A., Chohan, P. and Bloom, S. R. (1987a) Neuromedin U—a study of its distribution in the rat. Peptides 8, 779-84.
Domin, J., Steel, J. H., Adolphus, N., Burrin, J. M., Leonhardt, U., Polak, J. M. and Bloom, S. R. (1989a) The anterior pituitary content of neuromedin U-like immunoreactivity is altered by thyrotrophin-releasing hormone and thyroid hormone status in the rat. J Endocrinol 122, 471-6.
Domin, J., Yiangou, Y. G., Spokes, R. A., Aitken, A., Parmar, K. B., Chrysanthou, B. J. and Bloom, S. R. (1989b) The distribution, purification, and pharmacological action of an amphibian neuromedin U. J Biol Chem 264, 20881-5.
Favretti, F., De Luca, M., Segato, G., Busetto, L., Ceoloni, A., Magon, A. and Enzi, G. (2004) Treatment of morbid obesity with the Transcend Implantable Gastric Stimulator (IGS): a prospective survey. Obes Surg 14, 666-70.
Fujii, R., Hosoya, M., Fukusumi, S., Kawamata, Y., Habata, Y., Hinuma, S., Onda, H., Nishimura, O. and Fujino, M. (2000) Identification of neuromedin U as the cognate ligand of the orphan G protein-coupled receptor FM-3. J Biol Chem 275, 21068-74.
Funes, S., Hedrick, J. A., Yang, S., Shan, L., Bayne, M., Monsma, F. J. Jr and Gustafson, E. L. (2002) Cloning and characterization of murine neuromedin U receptors. Peptides 23, 1607-15.
Furness, J. B., Pompolo, S., Murphy, R. and Giraud, A. (1989a) Projections of neurons with neuromedin U-like immunoreactivity in the small intestine of the guinea-pig. Cell Tissue Res 257, 415-22.
Gardiner, S. M., Compton, A. M., Bennett, T., Domin, J. and Bloom, S. R. (1990) Regional hemodynamic effects of neuromedin U in conscious rats. Am J Physiol 258, R32-8.
Gartlon, J., Szekeres, P., Pullen, M., Sarau, H. M., Aiyar, N., Shabon, U., Michalovich, D., Steplewski, K., Ellis, C., Elshourbagy, N., Duxon, M., Ashmeade, T. E., Harrison, D. C., Murdock, P., Wilson, S., Ennaceur, A., Atkins, A., Heidbreder, C., Hagan, J. J., Hunter, A. J. and Jones, D. N. (2004) Localisation of NMU1R and NMU2R in human and rat central nervous system and effects of neuromedin-U following central administration in rats. Psychopharmacology (Berl) 177, 1-14.
Graham, E. S., Turnbull, Y., Fotheringham, P., Nilaweera, K., Mercer, J. G., Morgan, P. J. and Barrett, P. (2003a) Neuromedin U and Neuromedin U receptor-2 expression in the mouse and rat hypothalamus: effects of nutritional status. J Neurochem 87, 1165-73.
Guan, X. M., Yu, H., Jiang, Q., Van Der Ploeg, L. H. and Liu, Q. (2001a) Distribution of neuromedin U receptor subtype 2 mRNA in the rat brain. Brain Res Gene Expr Patterns 1, 1-4.
Hanada, R., Nakazato, M., Murakami, N., Sakihara, S., Yoshimatsu, H., Toshinai, K., Hanada, T., Suda, T., Kangawa, K., Matsukura, S, and Sakata, T. (2001) A role for neuromedin U in stress response. Biochem Biophys Res Commun 289, 225-8.
Hanada, R., Teranishi, H., Pearson, J. T., Kurokawa, M., Hosoda, H., Fukushima, N., Fukue, Y., Serino, R., Fujihara, H., Ueta, Y., Ikawa, M., Okabe, M., Murakami, N., Shirai, M., Yoshimatsu, H., Kangawa, K. and Kojima, M. (2004) Neuromedin U has a novel anorexigenic effect independent of the leptin signaling pathway. Nat Med 10, 1067-73.
Hanada, T., Date, Y., Shimbara, T., Sakihara, S., Murakami, N., Hayashi, Y., Kanai, Y., Suda, T., Kangawa, K. and Nakazato, M. (2003) Central actions of neuromedin U via corticotropin-releasing hormone. Biochem Biophys Res Commun 311, 954-8.
Hashimoto, T., Kurosawa, K. and Sakura, N. (1995) Structure-activity relationships of neuromedin U. II. Highly potent analogs substituted or modified at the N-terminus of neuromedin U-8. Chem Pharm Bull (Tokyo) 43, 1154-7.
Hashimoto, T., Masui, H., Uchida, Y., Sakura, N. and Okimura, K. (1991) Agonistic and antagonistic activities of neuromedin U-8 analogs substituted with glycine or D-amino acid on contractile activity of chicken crop smooth muscle preparations. Chem Pharm Bull (Tokyo) 39, 2319-22.
Hedrick, J. A., Morse, K., Shan, L., Qiao, X., Pang, L., Wang, S., Laz, T., Gustafson, E. L., Bayne, M. and Monsma, F. J. Jr (2000) Identification of a human gastrointestinal tract and immune system receptor for the peptide neuromedin U. Mol Pharmacol 58, 870-5.
Honzawa, M., Sudoh, T., Minamino, N., Kangawa, K. and Matsuo, H. (1990) Neuromedin U-like immunoreactivity in rat intestine: regional distribution and immunohistochemical study. Neuropeptides 15, 1-9.
Honzawa, M., Sudoh, T., Minamino, N., Tohyama, M. and Matsuo, H. (1987) Topographic localization of neuromedin U-like structures in the rat brain: an immunohistochemical study. Neuroscience 23, 1103-22.
Hosoya, M., Moriya, T., Kawamata, Y., Ohkubo, S., Fujii, R., Matsui, H., Shintani, Y., Fukusumi, S., Habata, Y., Hinuma, S., Onda, H., Nishimura, O. and Fujino, M. (2000) Identification and functional characterization of a novel subtype of neuromedin U receptor. J Biol Chem 275, 29528-32.
Howard, A. D., Wang, R., Pong, S. S., Mellin, T. N., Strack, A., Guan, X. M., Zeng, Z., Williams, D. L. Jr, Feighner, S. D., Nunes, C. N., Murphy, B., Stair, J. N., Yu, H., Jiang, Q., Clements, M. K., Tan, C. P., McKee, K. K., Hreniuk, D. L., McDonald, T. P., Lynch, K. R., Evans, J. F., Austin, C. P., Caskey, C. T., Van der Ploeg, L. H. and Liu, Q. (2000a) Identification of receptors for neuromedin U and its role in feeding. Nature 406, 70-4.
Ida, T., Mori, K., Miyazato, M., Egi, Y., Abe, S., Nakahara, K., Nishihara, M., Kangawa, K. and Murakami, N. (2005) Neuromedin S is a novel anorexigenic hormone. Endocrinology
Ivanov, T. R., Lawrence, C. B., Stanley, P. J. and Luckman, S. M. (2002) Evaluation of neuromedin U actions in energy homeostasis and pituitary function. Endocrinology 143, 3813-21.
Ivanov, T. R., Le Rouzic, P., Stanley, P. J., Ling, W. Y., Parello, R. and Luckman, S. M. (2004) Neuromedin U neurones in the rat nucleus of the tractus solitarius are catecholaminergic and respond to peripheral cholecystokinin. J Neuroendocrinol 16, 612-9.
Johnson, E. N., Appelbaum, E. R., Carpenter, D. C., Cox, R. F., Disa, J., Foley, J. J., Ghosh, S. K., Naselsky, D. P., Pullen, M. A., Sarau, H. M., Scheff, S. R., Steplewski, K. M., Zaks-Zilberman, M. and Aiyar, N. (2004) Neuromedin U elicits cytokine release in murine Th2-type T cell clone D10.G4.1. J Immunol 173, 7230-8.
Kage, R., O'Harte, F., Thim, L. and Conlon, J. M. (1991) Rabbit neuromedin U-25: lack of conservation of a posttranslational processing site. Regul Pept 33, 191-8.
Kojima, M., Haruno, R., Nakazato, M., Date, Y., Murakami, N., Hanada, R., Matsuo, H. and Kangawa, K. (2000) Purification and identification of neuromedin U as an endogenous ligand for an orphan receptor GPR66 (FM3). Biochem Biophys Res Commun 276, 435-8.
Kowalski, T. J., Spar, B. D., Markowitz, L., Maguire, M., Golovko, A., Yang, S., Farley, C., Cook, J. A., Tetzloff, G., Hoos, L., Del Vecchio, R. A., Kazdoba, T. M., McCool, M. F., Hwa, J. J., Hyde, L. A., Davis, H., Vassileva, G., Hedrick, J. A. and Gustafson, E. L. (2005) Transgenic overexpression of neuromedin U promotes leanness and hypophagia in mice. J Endocrinol 185, 151-64.
Kurosawa, K., Sakura, N. and Hashimoto, T. (1996) Structure-activity relationships of neuromedin U. III. Contribution of two phenylalanine residues in dog neuromedin U-8 to the contractile activity. Chem Pharm Bull (Tokyo) 44, 1880-4.
Lee, W. H., Liu, S. B., Shen, J. H., Jin, Y., Lai, R. and Zhang, Y. (2005) Identification and molecular cloning of a novel neuromedin U analog from the skin secretions of toad Bombina maxima. Regul Pept 129, 43-7.
Lin, K. S., Luu, A., Baidoo, K. E., Hashemzadeh-Gargari, H., Chen, M. K., Brenneman, K., Pili, R., Pomper, M., Carducci, M. A. and Wagner, H. N. Jr (2005) A new high affinity technetium-99m-bombesin analogue with low abdominal accumulation. Bioconjug Chem 16, 43-50.
Lo, G., Legon, S., Austin, C., Wallis, S., Wang, Z. and Bloom, S. R. (1992) Characterization of complementary DNA encoding the rat neuromedin U precursor. Mol Endocrinol 6, 1538-44.
Maderdrut, J. L., Lazar, G., Kozicz, T. and Merchenthaler, I. (1996) Distribution of neuromedin U-like immunoreactivity in the central nervous system of Rana esculenta. J Comp Neurol 369, 438-50.
Maggi, C. A., Patacchini, R., Giuliani, S., Turini, D., Barbanti, G., Rovero, P. and Meli, A. (1990) Motor response of the human isolated small intestine and urinary bladder to porcine neuromedin U-8. Br J Pharmacol 99, 186-8.
Malendowicz, L. K., Andreis, P. G., Markowska, A., Nowak, M., Warchol, J. B., Neri, G. and Nussdorfer, G. G. (1994a) Effects of neuromedin U-8 on the secretory activity of the rat adrenal cortex: evidence for an indirect action requiring the presence of the zona medullaris. Res Exp Med (Berl) 194, 69-79.
Malendowicz, L. K., Nussdorfer, G. G., Markowska, A., Tortorella, C., Nowak, M. and Warchol, J. B. (1994b) Effects of neuromedin U (NMU)-8 on the rat hypothalamo-pituitary-adrenal axis. Evidence of a direct effect of NMU-8 on the adrenal gland. Neuropeptides 26, 47-53.
Minamino, N., Kangawa, K., Honzawa, M. and Matsuo, H. (1988) Isolation and structural determination of rat neuromedin U. Biochem Biophys Res Commun 156, 355-60.
Minamino, N., Kangawa, K. and Matsuo, H. (1985a) Neuromedin U-8 and U-25: novel uterus stimulating and hypertensive peptides identified in porcine spinal cord. Biochem Biophys Res Commun 130, 1078-85.
Minamino, N., Sudoh, T., Kangawa, K. and Matsuo, H. (1985c) Neuromedins: novel smooth-muscle stimulating peptides identified in porcine spinal cord. Peptides 6 Suppl 3, 245-8.
Mondal, M. S., Date, Y., Murakami, N., Toshinai, K., Shimbara, T., Kangawa, K. and Nakazato, M. (2003) Neuromedin U acts in the central nervous system to inhibit gastric acid secretion via CRH system. Am J Physiol Gastrointest Liver Physiol 284, G963-9.
Mori, K., Miyazato, M., Ida, T., Murakami, N., Serino, R., Ueta, Y., Kojima, M. and Kangawa, K. (2005) Identification of neuromedin S and its possible role in the mammalian circadian oscillator system. EMBO J 24, 325-35.
Murphy, R., Turner, C. A., Furness, J. B., Parker, L. and Giraud, A. (1990) Isolation and microsequence analysis of a novel form of neuromedin U from guinea pig small intestine. Peptides 11, 613-7.
Nakahara, K., Kojima, M., Hanada, R., Egi, Y., Ida, T., Miyazato, M., Kangawa, K. and Murakami, N. (2004) Neuromedin U is involved in nociceptive reflexes and adaptation to environmental stimuli in mice. Biochem Biophys Res Commun 323, 615-20.
Nakazato, M., Hanada, R., Murakami, N., Date, Y., Mondal, M. S., Kojima, M., Yoshimatsu, H., Kangawa, K. and Matsukura, S. (2000) Central effects of neuromedin U in the regulation of energy homeostasis. Biochem Biophys Res Commun 277, 191-4.
Norlen, P., Bernsand, M., Konagaya, T. and Hakanson, R. (2001) ECL-cell histamine mobilization in conscious rats: effects of locally applied regulatory peptides, candidate neurotransmitters and inflammatory mediators. Br J Pharmacol 134, 1767-77.
O'Harte, F., Bockman, C. S., Abel, P. W. and Conlon, J. M. (1991a) Isolation, structural characterization and pharmacological activity of dog neuromedin U. Peptides 12, 11-5.
O'Harte, F., Bockman, C. S., Zeng, W., Abel, P. W., Harvey, S. and Conlon, J. M. (1991b) Primary structure and pharmacological activity of a nonapeptide related to neuromedin U isolated from chicken intestine. Peptides 12, 809-12.
Okimura, K., Sakura, N., Ohta, S., Kurosawa, K. and Hashimoto, T. (1992) Contractile activity of porcine neuromedin U-25 and various neuromedin U-related peptide fragments on isolated chicken crop smooth muscle. Chem Pharm Bull (Tokyo) 40, 1500-3.
Sakura, N., Kurosawa, K. and Hashimoto, T. (1995) Structure-activity relationships of neuromedin U. I. Contractile activity of dog neuromedin U-related peptides on isolated chicken crop smooth muscle. Chem Pharm Bull (Tokyo) 43, 1148-53.
Sakura, N., Kurosawa, K. and Hashimoto, T. (2000) Structure-activity relationships of neuromedin U. IV. Absolute requirement of the arginine residue at position 7 of dog neuromedin U-8 for contractile activity. Chem Pharm Bull (Tokyo) 48, 1166-70.
Sakura, N., Ohta, S., Uchida, Y., Kurosawa, K., Okimura, K. and Hashimoto, T. (1991) Structure-activity relationships of rat neuromedin U for smooth muscle contraction. Chem Pharm Bull (Tokyo) 39, 2016-20.
Salmon, A. L., Johnsen, A. H., Bienert, M., McMurray, G., Nandha, K. A., Bloom, S. R. and Shaw, C. (2000) Isolation, structural characterization, and bioactivity of a novel neuromedin U analog from the defensive skin secretion of the Australasian tree frog, Litoria caerulea. J Biol Chem 275, 4549-54.
Schubert, M. L. (2004) Gastric secretion. Curr Opin Gastroenterol 20, 519-25.
Simon, E. (2000) Interface properties of circumventricular organs in salt and fluid balance. News Physiol Sci 15, 61-67.
Steel, J. H., Van Noorden, S., Ballesta, J., Gibson, S. J., Ghatei, M. A., Burrin, J., Leonhardt, U., Domin, J., Bloom, S. R. and Polak, J. M. (1988) Localization of 7B2, neuromedin B, and neuromedin U in specific cell types of rat, mouse, and human pituitary, in rat hypothalamus, and in 30 human pituitary and extrapituitary tumors. Endocrinology 122, 270-82.
Sumi, S., Inoue, K., Kogire, M., Doi, R., Takaori, K., Suzuki, T., Yajima, H. and Tobe, T. (1987) Effect of synthetic neuromedin U-8 and U-25, novel peptides identified in porcine spinal cord, on splanchnic circulation in dogs. Life Sci 41, 1585-90.
Szekeres, P. G., Muir, A. I., Spinage, L. D., Miller, J. E., Butler, S. I., Smith, A., Rennie, G. I., Murdock, P. R., Fitzgerald, L. R., Wu, H., McMillan, L. J., Guerrera, S., Vawter, L., Elshourbagy, N. A., Mooney, J. L., Bergsma, D. J., Wilson, S. and Chambers, J. K. (2000) Neuromedin U is a potent agonist at the orphan G protein-coupled receptor FM3. J Biol Chem 275, 20247-50.
Tan, C. P., McKee, K. K., Liu, Q., Palyha, O. C., Feighner, S. D., Hreniuk, D. L., Smith, R. G. and Howard, A. D. (1998) Cloning and characterization of a human and murine T-cell orphan G-protein-coupled receptor similar to the growth hormone secretagogue and neurotensin receptors. Genomics 52, 223-9.
Timmermans, J. P., Scheuermann, D. W., Stach, W., Adriaensen, D. and De Groodt-Lasseel, M. H. (1990a) Distinct distribution of CGRP-, enkephalin-, galanin-, neuromedin U-, neuropeptide Y-, somatostatin-, substance P-, VIP- and serotonin-containing neurons in the two submucosal ganglionic neural networks of the porcine small intestine. Cell Tissue Res 260, 367-79.
Timmermans, J. P., Scheuermann, D. W., Stach, W., Adriaensen, D., De Groodt-Lasseel, M. H. and Polak, J. M. (1989) Neuromedin U-immunoreactivity in the nervous system of the small intestine of the pig and its coexistence with substance P and CGRP. Cell Tissue Res 258, 331-7.
Tsubota, Y., Kakimoto, N., Owada-Makabe, K., Yukawa, K., Liang, X. M., Mune, M. and Maeda, M. (2003) Hypotensive effects of neuromedin U microinjected into the cardiovascular-related region of the rat nucleus tractus solitarius. Neuroreport 14, 2387-90.
Westfall, T. D., McCafferty, G. P., Pullen, M., Gruver, S., Sulpizio, A. C., Aiyar, V. N., Disa, J., Contino, L. C., Mannan, I. J. and Hieble, J. P. (2002) Characterization of neuromedin U effects in canine smooth muscle. J Pharmacol Exp Ther 301, 987-92.
Yokota, M., Ozaki, Y., Sakamoto, F., Yamada, S., Saito, J., Fujihara, H. and Ueta, Y. (2004) Fos expression in CRF-containing neurons in the rat paraventricular nucleus after central administration of neuromedin U. Stress 7, 109-12.
Yu, X. H., Cao, C. Q., Mennicken, F., Puma, C., Dray, A., O'Donnell, D., Ahmad, S, and Perkins, M. (2003) Pro-nociceptive effects of neuromedin U in rat. Neuroscience 120, 467-74.

To assist in understanding the present invention, the following Examples are included. The examples illustrate the preparation of an NMX Peptide, FNX Peptide or NMX Receptor agonist (which includes derivatives) and the testing of these compounds in vitro and/or in vivo. The experiments relating to this invention should not be construed as specifically limiting the invention and variations thereof, now known or later developed, which would be within the purview of one skilled in the art.

### EXAMPLES

### Example 1

### Synthesis of the Caloric Intake Lowering Polypeptides

The polypeptides can be synthesized using standard polypeptide synthesis methods. Such methods are described below and in U.S. Pat. No. 6,610,824 and U.S. Pat. No. 5,686,411, the entireties of which are incorporated herein by reference.

The polypeptides are assembled on 4-(2′-4′-dimethoxyphenyl)-Fmoc aminomethyl phenoxy acetamide norleucine MBHA resin (Novabiochem, 0.55 mmole/g) using Fmoc-protected amino acids (Applied Biosystems, Inc.). In general, single-coupling cycles are used throughout the synthesis and Fast Moc (HBTU activation) chemistry is employed. However, at some positions coupling may be less efficient than expected and double couplings required. Deprotection (Fmoc group removal) of the growing peptide chain using piperidine likewise may not always be efficient and require double deprotection. Final deprotection of the completed peptide resin is achieved using a mixture of triethylsilane (0.2 mL), ethanedithiol (0.2 mL), anisole (0.2 mL), water (0.2 mL) and trifluoroacetic acid (15 mL) according to standard methods (Introduction to Cleavage Techniques, Applied Biosystems, Inc.) The peptides are precipitated in ether/water (50 mL) and centrifuged. The precipitate is reconstituted in glacial acetic acid and lyophilized. The lyophilized peptides are dissolved in water). Crude purity is then determined.

Solvent A (0.1% TFA in water) and Solvent B (0.1% TFA in ACN) are used in purification and analysis steps. Solutions containing the various polypeptides are applied to a preparative C-18 column and purified (10% to 40% Solvent B in Solvent A over 40 minutes). Purity of fractions are determined isocratically using a C-18 analytical column. Pure fractions are pooled furnishing the above-identified peptide. Analytical RP-HPLC (gradient 30% to 60% Solvent B in Solvent A over 30 minutes) of the lyophilized peptide to determine retention time.

Peptides are also synthesized as follows.

Polypeptides can be synthesized on a Pioneer continuous flow peptide synthesizer (Applied Biosystems) using PAL-PEG-PS resin (Applied Biosystems) with a loading of 0.2 mmol/g (0.25 mmole scale). Fmoc amino acid (4.0 eq, 1.0 mmol) residues were activated using 4.0 eq HBTU, 4.0 equivalent of HOBT, 8.0 equivalent of DIEA and coupled to the resin for 1 hour. The Fmoc group was removed by treatment with 20% (v/v) piperidine in dimethylformamide. Final deprotection and cleavage of the peptide from the solid support was performed by treatment of the resin with reagent B (93% TFA, 3% phenol, 3% water and 1% triisopropylsilane) for 2-3 hours. The cleaved peptide was precipitated using tert-butyl methyl ether, pelleted by centrifugation and lyophilized. The pellet was re-dissolved in water (10-15 mL), filtered and purified via reverse phase HPLC using a C-18 column and an acetonitrile/water gradient containing 0.1% TFA. The purified product was lyophilized and analyzed by ESI-LC/MS and analytical HPLC and were demonstrated to be pure (>98%). Mass results all agreed with calculated values.

Alternatively, peptides were assembled on a Symphony® peptide synthesizer (Protein Technologies, Inc., Woburn, Mass.) using Rink amide resin (Novabiochem, San Diego, Calif.) with a loading of 0.43-0.49 mmol/g at 0.050-0.100 mmol. Fmoc amino acid (Applied Biosystems, Inc. 5.0 eq, 0.250-0.500 mmol) residues were dissolved at a concentration of 0.10 M in 1-methyl-2-pyrrolidinone. All other reagents (HBTU, HOBT and N,N-diisopropylethylamine) were prepared as 0.55 M dimethylformamide solutions. The Fmoc protected amino acids were then coupled to the resin-bound amino acid using, HBTU (2.0 eq, 0.100-0.200 mmol), HOBT (1.8 eq, 0.090-0.18 mmol), N,N-diisopropylethylamine (2.4 eq, 0.120-0.240 mmol) for 2 hours. Following the last amino acid coupling, the peptide was deprotected using 20% (v/v) piperidine in dimethylformamide for 1 hour. Once peptide sequence is completed, the Symphony® peptide synthesizer is programmed to cleave the resin. Trifluoroacetic acid (TFA) cleavage of the peptide from resin was carried out using a reagent mixture composed of 93% TFA, 3% phenol, 3% water and 1% triisopropylsilane. The cleaved peptide was precipitated using tert-butyl methyl ether, pelleted by centrifugation and lyophilized. The pellet was dissolved in acetic acid, lyophilized and then dissolved in water, filtered and purified via reverse phase HPLC using a C18 column and an acetonitrile/water gradient containing 0.1% TFA. Analytical HPLC was used to assess purity of peptide and identity was confirmed by LC/MS and MALDI-MS.

### Example 2

### Effects on Caloric Intake

The effect of NMX Peptides, FNX Peptides or NMX Receptor agonists on food intake when systemically administered was investigated using an acute food intake assay. This assay measured food consumption in lean subjects. Lean, group-housed, overnight-fasted NIH/Swiss mice. All in vivo tests were performed with peripheral injections of peptide. When FN38 was administered systemically it dose-dependently inhibited food intake as described. The dose-response data are presented herein. The protocol is described below.

Female NIH/Swiss mice (8-24 weeks old) were group housed with a 12:12 hour light:dark cycle, with lights on at 0300. Water and a standard pelleted mouse chow diet are available ad libitum, except as noted. Animals are fasted starting at approximately 1530 hrs, 1 day prior to experiment.

At time=0 min, all animals are given an intraperitoneal injection of vehicle or polypeptide in a volume of 200 uL/mouse and immediately given a pre-weighed amount (10-15 g) of the standard chow. Food is removed and weighed at 30, 60, and 120 minutes to determine the amount of food consumed. The effects of treatment on food intake are expressed as % change relative to control.

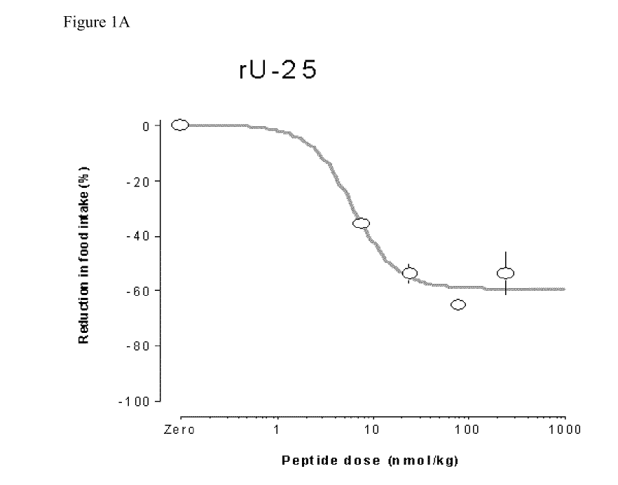

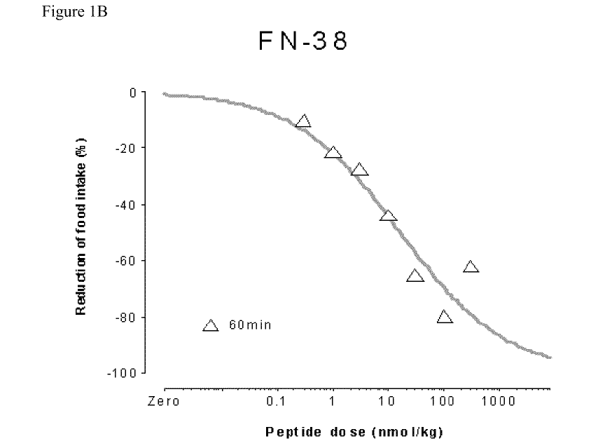

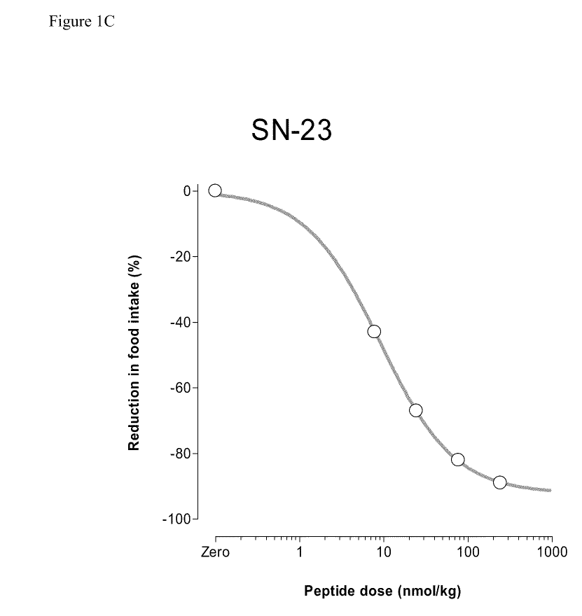

As can be seen in FIGS. 1A, 1B and 1C, various compounds dose-dependently reduced food intake at 30 minutes post injection.

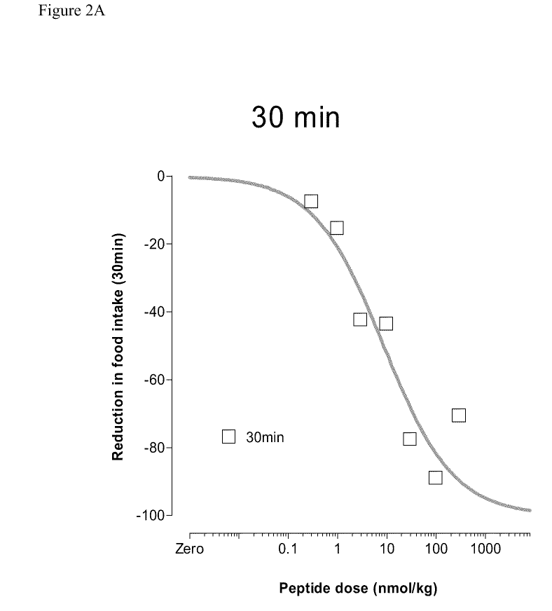

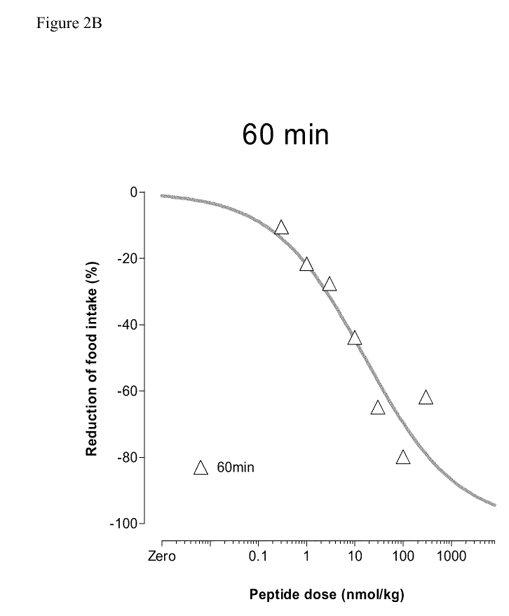

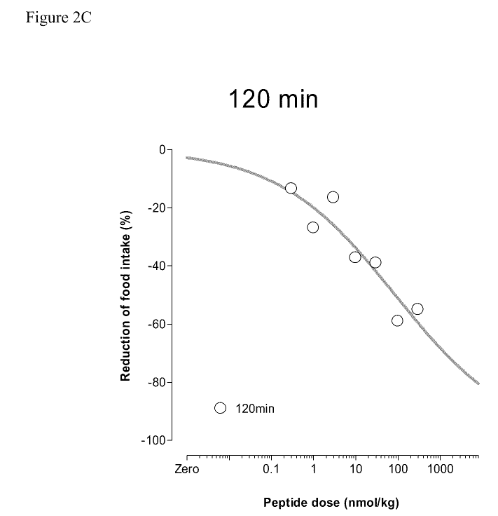

The table below depicts reduced food intake with FN38 administered peripherally (intraperitoneal injection) at doses indicated. The data at time points 30, 60, and 120 minutes represents the percent decrease in cumulative food intake compared to the vehicle. (see also FIGS. 2A, 2B and 2C):

| | | | Concentration | |
|---|---|---|---|---|
| Activity | Calculation | Timepoint min | Doses | C_unit |
| 0 | AUC | 120 | 0.3 | nmol/kg |
| −23 | AUC | 120 | 1 | nmol/kg |
| −25 | AUC | 120 | 3 | nmol/kg |
| −48 | AUC | 120 | 10 | nmol/kg |
| −58 | AUC | 120 | 30 | nmol/kg |
| −72 | AUC | 120 | 100 | nmol/kg |
| −60 | AUC | 120 | 300 | nmol/kg |
| −46 | AUC | 120 | 1000 | ug/kg |

The ED50 for effect of FN38 on food intake over 30 min was 8.6 nmol/kg. Rat U-23 was less effective (−60% change in intake vs. >−90% for FN38 and SN23), but was equipotent with FN38 (ED50 6.2 nmol/kg). The frog homolog SN23 was fully effective and equipotent (ED50 9.0 nmol/kg).

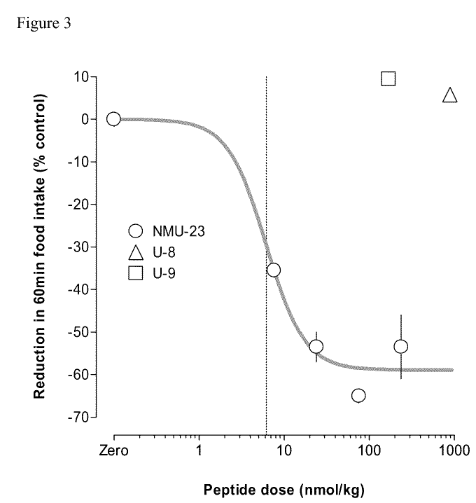

In contrast, despite their description as potent NMU1R and NMU2R agonists reported in the literature, neither porcine U-8 nor U-9 (GYFLFRPRNamide) (SEQ ID NO.: 11) were active in food intake assays herein (see FIG. 3, and data not shown). U-8 was inactive at doses between 100-1000 μg/kg (up to 900 nmol/kg), i.e. 100× higher than the ED50's for FN-38, rNMU-23 or SN-23. U-9 was inactive at doses up to 170 nmol/kg, 20× higher than the ED50's of the longer agonists. The difference in potency between U-8 and rNMU-23 was at least 1000-fold.

The absence of effect of U-8/U-9 might be explained by some specific degradation/disablement of those peptides that does not occur with the longer peptides, such as an increased susceptibility to peptidase cleavage. However, based on published reports, accelerated degradation of U-8 would be unlikely to account for its lack of effect in vivo, since it has been observed that for several in vivo hemodynamic effects U-8 was more potent than U-25 in dogs following intravenous bolus injection (Gardiner et al. 1990; Sumi et al. 1987). However, differential degradation assessed by the present inventors using serial MS analyses in vitro in plasma suggests a rapid degradation. Doses at which we observed herein an absence of anorectic effect (in mice) were 1-2 orders of magnitude higher than the lower U8 dose for maximal hemodynamic effects in dogs (Sumi et al. 1987). In the rat, U-25 and U-8 bolus doses (0.1 and 1.0 nmol) or infusions (1 and 10 nmol/h) each exerted potent constrictor effects on the superior mesenteric vascular bed, and even though U-25 was generally more potent than U-8, the difference was generally not more than 3-fold (Gardiner et al. 1990).

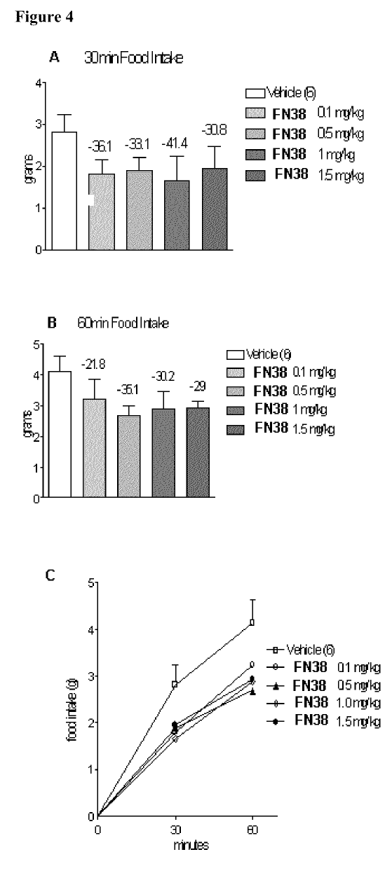

FN38 also demonstrated the effect of inhibiting food intake in over-night fasted rats when given intraperitoneally. See FIGS. 4A, 4B and 4C. FN38 in vehicle (10% DMSO in saline) was administered at 0.1 to 1.5 mg/kg. The food intake in grams and as a percent of vehicle are presented in FIGS. 4A and 4B at 30 and 60 minutes, respectively. FIG. 4C presents the same data plotted as grams of food consumed (intake) versus time. The results cannot be attributed to a locomotor effect (data not shown).

As noted herein various NMX Peptides were tested and found to have an effect on reducing food intake when administered systemically. Human neuromedin U-25, sequence FRVDEEFQSPFASQSRGYFLFRPRN-NH2 (SEQ ID NO.: 12) reduced food intake by at least 91%. Rat U-23, sequence YKVNEYQGPVAPSGGFFLFRPRN-NH2 (SEQ ID No.: 10) administered i.p. to mice reduced food intake by up to 66%. The ED50 was 6.2 nmol/kg at 30 min. If this dose was instantaneously distributed into the extracranial space, U-25 concentration would be 6.2 nM. This can be compared to the 10 μg ICV dose required to effect a similar 30% reduction in food intake in rats (Howard et al. 2000a). If distributed instantaneously throughout the 2 cc intracranial space in the rat (literature value for 400 g rats), this 10 μg dose of U-25 would generate a concentration of 1.9 μM. That is, on a presumed concentration basis, U-25 was ˜300-fold less potent administered centrally (i.c.v.) than when administered peripherally. Conversely, U25 was 300 times more potent administered peripherally than centrally. The results herein are consistent with receptors of relevance to feeding being located outside rather than inside the blood-brain-barrier, in contrast to current views in the literature.

Frog NMU homolog (SN23) was also an effective anorexigen, reducing food intake by up to 95%. Interestingly, an FN38-SN23 chimera in which the 15 amino acid FN38 N-terminal preceded the SN23 N-terminal (Compound 165063) reduced food intake by at least 89%, whereas FN38 evoked a reduction by 64%.

Neuromedin S, also referred to as NMS or IN33 (Compound No. 165050; ILQRGSGTAAVDFTKKDHTATWGRPFFLFRPRN-NH2 (SEQ ID No. 71) shares a 7 amino acid N-terminal with NMU's, FN-38, U-23, U-25, U-8 and U-9, and was also effective in reducing food intake (by at least −64%).

### Example 3

### Activity of Compounds on Body Weight and Food Intake in Obese Animals

The effect of FN38 and related compounds on body weight and food intake in obese subjects was investigated. Mice having diet induced obesity (DIO) were used. All in vivo tests were performed with peripheral injections of peptide. When injected peripherally, FN38 dose-dependently reduced body weight and inhibited food intake as described herein.

4-week-old male C57BL/6 mice were group housed with water and a standard pelleted mouse chow diet available ad libitum, except as noted, and were maintained on a on a high fat diet (58% kcal from fat) for 4 weeks prior to the experiment. At the end of fattening period, osmotic pumps were implanted interscapularly under anesthesia. Mice received pumps continuously delivering vehicle (50% DMSO in water or saline) or polypeptide at the dose indicated. Food intake and body weight measurements were obtained weekly.

FN38 decreased body weight by 6.5% at 2d, and by 4.3% at 7d in the mouse DIO. SN-23 did not have a long acting effect in mouse DIO. Additional NMX Peptides and FNX Peptides provided desirable weight reduction as indicated in the tables below.

| | | | | d 2 | d 7 | d 14 | |
|---|---|---|---|---|---|---|---|
| Compd | FI | ED50 at | | BWt | BWt | BWt | |
| # | (60′, 200) | t = 60 | | loss | loss | loss | BBM |
| 163291 | −75 | | | | | | |
| 163293 | −37.5 | 17 | μg/kg | — | — | 3.7 | |
| 163357 | 26 | | | | | | |
| 163452 | −88 | 22 | μg/kg | — | — | 1.5 | |
| 163661 | 0 | 4 | nmol/kg | 6.5 | 4.3 | — | 62 |
| 165063 | −88 | 6 | nmol/kg | — | 5.1 | 8.6 | 73 |
| 165061 | 0 | | | | | | 57 |
| 165062 | 0 | | | | | | 40 |
| 165054 | −65 | | | | | | 13 |

Both rat NMU-23 and tree frog SN-23, at 75 nmol/kg/day in the DIO assay decreased body weight gain and food intake, however, not as effectively as FN38.

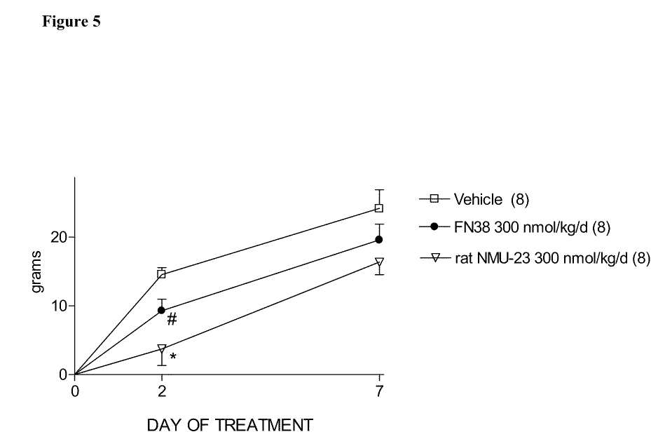

FIG. 5 depicts the reduction of cumulative weight gained by peripherally administered FN38 amide and rat NMU-23 amide in rats with diet induced obesity (rat DIO). Both compounds were administered subcutaneously continuously by pump for seven days at the rate and dosage indicated.

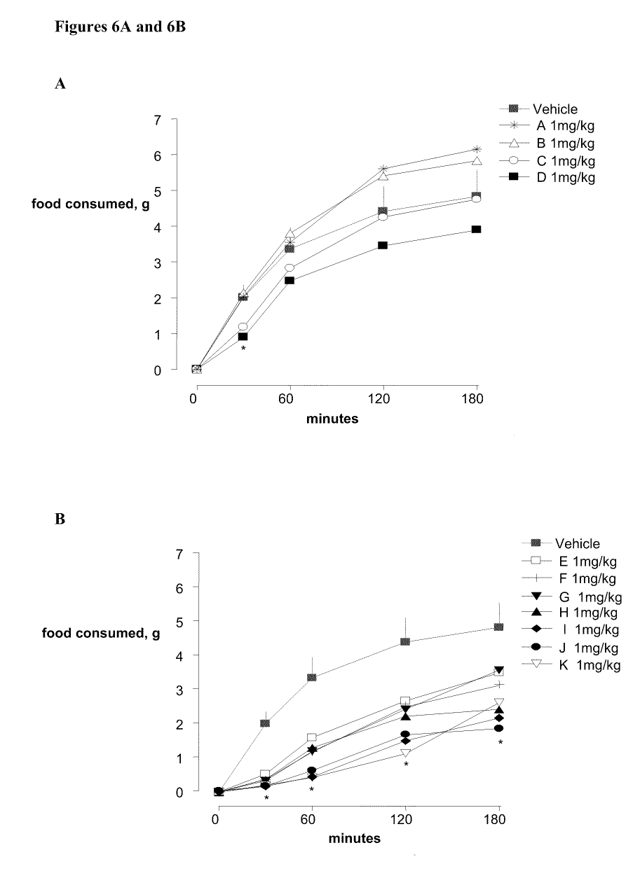

FIGS. 6A and B reduction of cumulative food intake by exemplary FNX Peptides in mouse DIO. The activities of the following compounds are shown: A, [MimicB36] FN38; B, [His37] FN38; C,

[His35] FN38; D, [Phe33] FN38; E, [Val33] FN38; F, Des-(Ser6,Lys7,Thr8,Gln9) FN38; G, Des-Val16,Val17-FN38; H, [Phe31] FN38; I, Des-(Val16,Val17,Phe24,Ala25,Ser26,Gln27) FN38; J, Des-(Phe1,Leu2,Phe3,His4)FN38; and K, Des-(Phe24,Ala25,Ser26,Gln27)-FN38. Compounds A, B, C, and D were either not active or weakly active in this particular assay. Points represent mean±sd of n=4 cages (3 mice/cage). Peptide was injected IP at t=0. Food was introduced immediately after injection and amount consumed measured at t=30, 60, 120, and 180 min. In the figure, * p<0.05 vs. vehicle control; ANOVA, Dunnett's test.

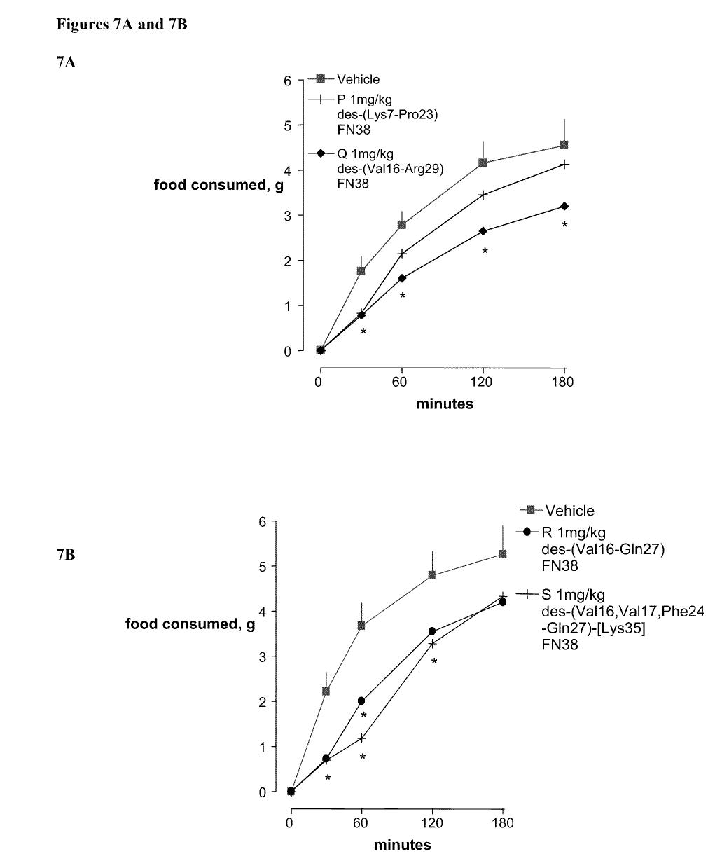

FIGS. 7A and 7B show reduction of cumulative food intake by exemplary FNX Peptides in mouse DIO: P, des-(Lys7-Pro23) FN38; Q, des-(Val16-Arg29); R, des-(Val16-Gln27) FN38; and S, des-(Val 16,Val 17,Phe24-Gln27)-[Lys35] FN38.

### Example 4

### Peptidase Stability of the Compounds

Some exemplary NMX Peptides and FNX Peptides were tested for stability in a brush border membrane (BBM) assay as described herein. Results are shown in the tables above. To determine the enzymatic stability of peptide hormones peptides are incubated with a preparation of human brush border membranes extract (e.g., kidney), and the stability of the peptide is determined by measuring concentration of the intact peptide at specific intervals. The brush border membrane extract contains dipeptidyl peptidase IV (DPP-IV), neutral endopeptidases, peptidyl-dipeptidase A, carboxypeptidases and aminopeptidases. These are the primary enzymes that degrade peptides in vivo and are found in the kidneys, liver, lungs and pancreas. Resistance against these human proteases would increase peptide half-life.

In the BBM assay test peptide was subjected to digestion over a period of five (5) hours with human brush border membrane extracts, e.g. kidney, at 37° C. At desired timepoints digestion was stopped by addition of quench solution, typically 50% ACN, 1% FA. After centrifugation to remove membrane debris, supernatant was subjected to mass spectrometry using a selected ion scan for the intact molecule of interest. Values are expressed as a percent of stable. A value of at least 80 or greater is regarded as an extremely stable molecule. A value of at least 70 or greater is a molecule with significant stability.

It was also determined that FN38 is resistant to DPP-IV degradation with a similar resistance as exendin-4, with essentially 100% intact after 50 minutes of contact with DPP-IV under conditions in which GLP-1 is completely cleaved to its inactive metabolite. Peptide and peptidase were incubated in 25 mM HEPES buffer at 37 degrees C. at 10 mM peptide.

While the present invention has been described in terms of examples and embodiments, it is understood that variations and modifications will occur to those skilled in the art, which are intended to be covered by the claims. All documents described herein are incorporated by reference in their entirety.

What is claimed is:

1. An polypeptide comprising an FNX Peptide, wherein the FNX Peptide comprises an amino acid sequence of formula (I): F1-P, where F1-P is a combination of an F1 segment and a P segment, where P is an octapeptide capable of providing, when attached to F1 and systemically delivered, suppression of food intake, reduction of body weight, and/or induction of a satiety signal or a distension signal, and wherein F1 is a des-octapeptide portion of an FN38 or analog, derivative or chimera thereof, which enhances or enables P activity, and with the proviso that excluded from F1-P are the polypeptides corresponding to GenBank Accession Number AJ510133 (human), CAD52851 (rat), CAD52850 (frog) and chicken FN38.

2. The polypeptide of claim 1, wherein the FNX Peptide has a biological activity which comprises an ability to cause an inhibition or reduction in food, nutrient or caloric intake or availability or a reduction or suppression of appetite, when administered peripherally to a subject.

3. The polypeptide of claim 2, wherein the P region comprises an amino acid sequence of an octapeptide of a native neuromedin U peptide or an analog, derivative or active fragment thereof that corresponds to the C-terminal octapeptide of FN38.

4. The polypeptide of claim 3, wherein the octapeptide is native human YFLFRPRN (SEQ ID NO. 11), Zebra fish YFLYRPRN (SEQ ID NO. 72), frog YFVFRPRN (SEQ ID NO. 73), rat FFLFRPRN (SEQ ID NO. 74) or toad FFLFRPRN (SEQ ID NO. 75), or an analog or derivative thereof.

5. The polypeptide of claim 4, wherein the octapeptide P is FFFYHPHN (SEQ ID NO. 27), FFFFRPRN (SEQ ID NO. 28), FFFFKHHN (SEQ ID NO. 29), or FFFFK(beta turn mimic)HN (SEQ ID NO. 30).

6. The polypeptide of claim 1, wherein the F1 region is a des-octapeptide (des-P segment) of a native FN38 or an analog or derivative thereof.

7. The polypeptide of claim 6, wherein the native F1 sequence is FLFHYSKTQKLGKSNVVEELQSPFASQSR (SEQ ID NO. 76) or the F1 region is a des-octapeptide selected from FN38(des 16,17), FN38(des 24-27), FN38(des 16,17)(des 24-27), FN38(des 1-4), FN38(des 6-9), FN38(des 13-19), FN38(des 2-8), Des-(Lys7-Pro23)FN38, Des-(Val 16-Arg29)FN38, Des-(Val 16-Gln27)FN38, and Des-(Val 16,Val 17,Phe24-Gln27)[K35]FN38, or an analog or derivative thereof.

8. The polypeptide of claim 6, wherein the F1 region is FLFHYS (SEQ ID NO. 77).

9. The polypeptide of claim 8, wherein the FNX Peptide is FLFHYSGYFLFRPRN (SEQ ID NO. 65) or its amide, or an analog or derivative thereof.

10. A polypeptide comprising an FNX Peptide, wherein the FNX Peptide comprises an amino acid sequence of formula (II): F2-P, where P is an octapeptide capable of providing, when attached to the F2 portion and systemically delivered, suppression of food intake, reduction of body weight, and/or induction of a satiety signal or distension signal, and wherein F2 is a des-octapeptide portion of a chimera of FN38 and SN23 or analog or derivative thereof, which enhances or enables P activity.

11. The polypeptide of claim 10 wherein in F2 comprises the des-octapeptide region of FN38(1-15)-SN23 or an analog or derivative thereof.

12. The polypeptide of claim 11 wherein F2-P comprises FN38(1-15)-SN23.

13. The polypeptide of claim 10, wherein the F2 is selected from the F2 region of FN38(1-15)-SN23-Y31F, FN38(1-15)-SN23-F34V, FN38(1-15)-SN23-L33F, FN38(1-15)-SN23-R35H, FN38(1-15)-SN23-R37H, or FN38(1-15)-SN23-P36beta turn mimic, or an analog or derivative thereof.

14. The polypeptide of claim 10 wherein the octapeptide P is native human YFLFRPRN (SEQ ID NO. 11), Zebra fish YFLYRPRN (SEQ ID NO. 72), frog YFVFRPRN (SEQ ID NO. 73), rat FFLFRPRN (SEQ ID NO. 74) or toad FFLFRPRN (SEQ ID NO. 75), or an analog or derivative thereof.

15. The polypeptide of claim 14, wherein the octapeptide P is FFFYHPHN (SEQ ID NO. 27), FFFFRPRN (SEQ ID NO. 28), FFFFKHHN (SEQ ID NO. 29), or FFFFK(beta turn mimic)HN (SEQ ID NO. 30).

16. A polypeptide of claim 1 wherein the FNX Peptide, including analogs, derivatives and active fragment thereof, has at least 75% amino acid identity to any native FNX Peptide amino acid sequence.

17. A composition comprising a polypeptide according to claim 1 or a pharmaceutically acceptable salt thereof, and optionally a pharmaceutically acceptable carrier.

18. A method of treating or preventing a condition or disease that can be alleviated by reducing caloric or nutrient intake or availability in a subject in need thereof comprising administering to the subject an amount of an NMX Peptide, FNX Peptide or NMX Receptor agonist therapeutically effective to reduce caloric or nutrient intake or availability.

19. The method according to claim 18, wherein the condition or disease is obesity, insulin resistance, metabolic syndrome or diabetes mellitus.

20. The method according to claim 19, wherein the composition is administered peripherally.


SEQUENCE LISTING (77 sequences)

SEQ ID NO: 1 - protein, 45 aa, artificial
  synthetic
FLFHYSKTQKLGKSNVVEEFQSPFASQSRGYFLFRPRNGRRSAGF

SEQ ID NO: 2 - protein, 38 aa, artificial
  synthetic
FLFHYSKTQKLGKSNSDEEVQVPGGVISNGYFLFRPRN

SEQ ID NO: 3 - protein, 23 aa, tree frog
SDEEVQVPGGVISNGYFLFRPRN

SEQ ID NO: 4 - protein, 38 aa, Homo sapiens
FLFHYSKTQKLGKSNVVEELQSPFASQSRGYFLFRPRN

SEQ ID NO: 5 - protein, 8 aa, artificial
  synthetic
YFLFRPRN

SEQ ID NO: 6 - protein, 30 aa, artificial
  synthetic
FLFHYSKTQKLGKSNSDEEVQVPGGVISNG

SEQ ID NO: 7 - protein, 36 aa, Rattus rattus
FLFHYSKTQKLGNSNVVEYQGPVAPSGGFFLFRPRN

SEQ ID NO: 8 - protein, 38 aa, Chicken
FLFHYSKTHDSGNSDVREDLQGTGGIQSRGYFFFRPRN

SEQ ID NO: 9 - protein, 38 aa, tree frog
FLFHYSKSHDSGNSDITEEVQVPGGVISNGYFLFRPRN

SEQ ID NO: 10 - protein, 23 aa, rattus rattus
YKVNEYQGPVAPSGGFFLFRPRN

SEQ ID NO: 11 - protein, 9 aa, Homo sapiens
GYFLFRPRN

SEQ ID NO: 12 - protein, 25 aa, Homo sapiens
FRVDEEFQSPFASQSRGYFLFRPRN

SEQ ID NO: 13 - protein, 30 aa, artificial; identical to SEQ ID NO: 6
  synthetic

SEQ ID NO: 14 - protein, 30 aa, artificial
  synthetic
FLFHYSKTQKLGKSNVVEELQSPFASQSRG

SEQ ID NO: 15 - protein, 38 aa, artificial
  synthetic
FLFHYSKTQKLGKSNVVEELQSPFASQSRGFFLFRPRN

SEQ ID NO: 16 - protein, 38 aa, artificial
  synthetic
FLFHYSKTQKLGKSNVVEELQSPFASQSRGYFLVRPRN

SEQ ID NO: 17 - protein, 38 aa, artificial
  synthetic
FLFHYSKTQKLGKSNVVEELQSPFASQSRGYFFFRPRN

SEQ ID NO: 18 - protein, 38 aa, artificial
  synthetic
FLFHYSKTQKLGKSNVVEELQSPFASQSRGYFLFHPRN

SEQ ID NO: 19 - protein, 38 aa, artificial
  synthetic
FLFHYSKTQKLGKSNVVEELQSPFASQSRGYFLFRPHN

SEQ ID NO: 20 - protein, 38 aa, artificial
  synthetic
FLFHYSKTQKLGKSNVVEELQSPFASQSRGYFLFRXRN

SEQ ID NO: 21 - protein, 38 aa, artificial
  synthetic
FLFHYSKTQKLGKSNSDEEVQVPGGVISNGFFLFRPRN

SEQ ID NO: 22 - protein, 38 aa, Artificial
  synthetic
FLFHYSKTQKLGKSNSDEEVQVPGGVISNGYFLVRPRN

SEQ ID NO: 23 - protein, 38 aa, artificial
  synthetic
FLFHYSKTQKLGKSNSDEEVQVPGGVISNGYFFFRPRN

SEQ ID NO: 24 - protein, 38 aa, artificial
  synthetic
FLFHYSKTQKLGKSNSDEEVQVPGGVISNGYFLFHPRN

SEQ ID NO: 25 - protein, 38 aa, artificial
  synthetic
FLFHYSKTQKLGKSNSDEEVQVPGGVISNGYFLFRPHN

SEQ ID NO: 26 - protein, 38 aa, artificial
  synthetic
FLFHYSKTQKLGKSNSDEEVQVPGGVISNGYFLFRXRN

SEQ ID NO: 27 - protein, 8 aa, artificial
  synthetic
FFFYHPHN

SEQ ID NO: 28 - protein, 8 aa, artificial
  synthetic
FFFFRPRN

SEQ ID NO: 29 - protein, 8 aa, artificial
  synthetic
FFFFKHHN

SEQ ID NO: 30 - protein, 8 aa, artificial
  synthetic
FFFFKXHN

SEQ ID NO: 31 - protein, 38 aa, artificial
  synthetic
FLFHYSKTQKLGKSNVVEELQSPFASQSRGFFFYHPHN

SEQ ID NO: 32 - protein, 38 aa, artificial
  synthetic
FLFHYSKTQKLGKSNVVEELQSPFASQSRGFFFFRPRN

SEQ ID NO: 33 - protein, 38 aa, artificial
  synthetic
FLFHYSKTQKLGKSNVVEELQSPFASQSRGFFFFKHHN

SEQ ID NO: 34 - protein, 38 aa, artificial
  synthetic
FLFHYSKTQKLGKSNVVEELQSPFASQSRGFFFFKXHN

SEQ ID NO: 35 - protein, 38 aa, artificial
  synthetic
FLFHYSKTQKLGKSNSDEEVQVPGGVISNGFFFYHPHN

SEQ ID NO: 36 - protein, 38 aa, artificial
  synthetic
FLFHYSKTQKLGKSNSDEEVQVPGGVISNGFFFFRPRN

SEQ ID NO: 37 - protein, 38 aa, artificial
  synthetic
FLFHYSKTQKLGKSNSDEEVQVPGGVISNGFFFFKHHN

SEQ ID NO: 38 - protein, 38 aa, artificial
  synthetic
FLFHYSKTQKLGKSNSDEEVQVPGGVISNGFFFFKXHN

SEQ ID NO: 39 - protein, 36 aa, artificial
  synthetic
FLFHYSKTQKLGKSNEELQSPFASQSRGYFLFRPRN

SEQ ID NO: 40 - protein, 34 aa, artificial
  synthetic
FLFHYSKTQKLGKSNVVEELQSPSRGYFLFRPRN

SEQ ID NO: 41 - protein, 32 aa, artificial
  synthetic
FLFHYSKTQKLGKSNEELQSPSRGYFLFRPRN

SEQ ID NO: 42 - protein, 34 aa, artificial
  synthetic
YSKTQKLGKSNVVEELQSPFASQSRGYFLFRPRN

SEQ ID NO: 43 - protein, 34 aa, artificial
  synthetic
FLFHYKLGKSNVVEELQSPFASQSRGYFLFRPRN

SEQ ID NO: 44 - protein, 31 aa, artificial
  synthetic
FLFHYSKTQKLGLQSPFASQSRGYFLFRPRN

SEQ ID NO: 45 - protein, 31 aa, artificial
  synthetic
FQKLGKSNVVEELQSPFASQSRGYFLFRPRN

SEQ ID NO: 46 - protein, 21 aa, artificial
  synthetic
FLFHYSFASQSRGYFLFRPRN

SEQ ID NO: 47 - protein, 24 aa, artificial
  synthetic
FLFHYSKTQKLGKSNGYFLFRPRN

SEQ ID NO: 48 - protein, 26 aa, artificial
  synthetic
FLFHYSKTQKLGKSNSRGYFLFRPRN

SEQ ID NO: 49 - protein, 32 aa, artificial
  synthetic
FLFHYSKTQKLGKSNEELQSPSRGYFLFKPRN

SEQ ID NO: 50 - protein, 15 aa, artificial
  synthetic
FLFHYSGYFLFRPRN

SEQ ID NO: 51 - protein, 29 aa, artificial
  synthetic
KLGKSNVVEELQSPFASQSRGYFLFRPRN

SEQ ID NO: 52 - protein, 28 aa, artificial
  synthetic
YSKTQKLGKSNEELQSPSRGYFLFRPRN

SEQ ID NO: 53 - protein, 15 aa, artificial
  synthetic
FLFHYSGYFLFRXRN

SEQ ID NO: 54 - protein, 15 aa, artificial
  synthetic
FLFHYSGFFLFRXHN

SEQ ID NO: 55 - protein, 15 aa, artificial
  synthetic
FLFHYSGYFLFKPRN

SEQ ID NO: 56 - protein, 15 aa, artificial
  synthetic
FLFHYSGFFLFRPRN

SEQ ID NO: 57 - protein, 15 aa, artificial
  synthetic
FLFHYSGFFLFKPRN

SEQ ID NO: 58 - protein, 36 aa, artificial
  synthetic
FLFHYSKTQKLGKSNEEVQVPGGVISNGYFLFRPRN

SEQ ID NO: 59 - protein, 34 aa, artificial
  synthetic
FLFHYSKTQKLGKSNSDEEVQVPSNGYFLFRPRN

SEQ ID NO: 60 - protein, 32 aa, artificial
  synthetic
FLFHYSKTQKLGKSNEEVQVPSNGYFLFRPRN

SEQ ID NO: 61 - protein, 34 aa, artificial
  synthetic
YSKTQKLGKSNSDEEVQVPGGVISNGYFLFRPRN

SEQ ID NO: 62 - protein, 34 aa, artificial
  synthetic
FLFHYKLGKSNSDEEVQVPGGVISNGYFLFRPRN

SEQ ID NO: 63 - protein, 31 aa, artificial
  synthetic
FLFHYSKTQKLGVQVPGGVISNGYFLFRPRN

SEQ ID NO: 64 - protein, 31 aa, artificial
  synthetic
FQKLGKSNSDEEVQVPGGVISNGYFLFRPRN

SEQ ID NO: 65 - protein, 15 aa, artificial
  synthetic
FLFHYSGYFLFRPRN

SEQ ID NO: 66 - protein, 15 aa, artificial
  synthetic
FLFHYSGYFLFRXRN

SEQ ID NO: 67 - protein, 15 aa, artificial
  synthetic
FLFHYSGFFLFRXHN

SEQ ID NO: 68 - protein, 15 aa, artificial
  synthetic
FLFHYSGYFLFKPRN

SEQ ID NO: 69 - protein, 15 aa, artificial
  synthetic
FLFHYSGFFLFRPRN

SEQ ID NO: 70 - protein, 15 aa, artificial
  synthetic
FLFHYSGFFLFKPRN

SEQ ID NO: 71 - protein, 33 aa, Homo sapiens
ILQRGSGTAAVDFTKKDHTATWGRPFFLFRPRN

SEQ ID NO: 72 - protein, 9 aa, zebra fish
GYFLYRPRN

SEQ ID NO: 73 - protein, 9 aa, frog
GYFVFRPRN

SEQ ID NO: 74 - protein, 9 aa, rat
GFFLFRPRN

SEQ ID NO: 75 - protein, 9 aa, toad
PFFLFRPRN

SEQ ID NO: 76 - protein, 29 aa, artificial
  synthetic
FLFHYSKTQKLGKSNVVEELQSPFASQSR

SEQ ID NO: 77 - protein, 6 aa, artificial
  synthetic
FLFHYS